(19) United States
(12) Patent Application Publication
(10) Pub. No.: US 2012/0164188 A1
(43) Pub. Date: Jun. 28, 2012

(54) PALIPERIDONE OR A PHARMACEUTICALLY ACCEPTABLE SALT THEREOF SUBSTANTIALLY FREE OF IMPURITIES

(75) Inventors: Girish Dixit, Uttar Pradesh (IN); Anil Shahaji Khile, Maharashtra (IN); Jayesh Laljibhai Patel, Gujarat (IN); Nitin Sharadchandra Pradhan, Maharashtra (IN)

(73) Assignee: ACTAVIS GROUP PTC EHF, Hafnarfjorour (IS)

(21) Appl. No.: 13/381,716

(22) PCT Filed: Sep. 10, 2010
(86) PCT No.: PCT/IB10/02568

§ 371 Date: Mar. 15, 2012

(30) Foreign Application Priority Data
Sep. 10, 2009 (IN) 2188/CHE/2009

(51) Int. Cl. A61K 31/519; A61K 9/14; A61P 25/18; C07D 471/04

(52) U.S. Cl. 424/400; 544/282; 514/259.41

(57) ABSTRACT

Provided herein are impurities of paliperidone, 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one (methanoyl impurity), 3-[2-[4-(6-fluoro-1,2-benzisoxazol-3-yl)-1-piperidinyl]ethyl]-2-methyl-4H-pyrido[1,2-a]pyrimidin-4-one (dehydroxy impurity) and 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione (9-keto impurity), and processes for preparing and isolating thereof. Provided further herein is a highly pure paliperidone or a pharmaceutically acceptable salt thereof substantially free of methanoyl, dehydroxy and 9-keto impurities, process for the preparation thereof, and pharmaceutical compositions comprising highly pure paliperidone or a pharmaceutically acceptable salt thereof substantially free of methanoyl, dehydroxy and 9-keto impurities. Provided also herein are improved and efficient processes for preparing paliperidone intermediates.

### CROSS REFERENCE TO RELATED APPLICATION

This application claims the benefit of priority to Indian provisional application No. 2188/CHE/2009, filed on Sep. 10, 2009, which is incorporated herein by reference in its entirety.

### FIELD OF THE DISCLOSURE

Disclosed herein are impurities of paliperidone, and processes for the preparation and isolation thereof. Disclosed further herein is a highly pure paliperidone or a pharmaceutically acceptable salt thereof substantially free of impurities, process for the preparation thereof, and pharmaceutical compositions comprising highly pure paliperidone or a pharmaceutically acceptable salt thereof substantially free of impurities. Disclosed also herein are improved and efficient processes for preparing paliperidone intermediates.

### BACKGROUND

U.S. Pat. Nos. 4,804,663 and 5,158,952 disclose a variety of 3-piperidinyl-1,2-benzisoxazole derivatives, processes for their preparation, pharmaceutical compositions comprising the derivatives, and methods of use thereof. These compounds have long-acting antipsychotic properties and are useful in the treatment of warm-blooded animals suffering from psychotic diseases. Among them, paliperidone, (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, is an antipsychotic agent and indicated for the both acute (short-term) and maintenance (long-term) treatment of schizophrenia. Paliperidone is represented by the following structural formula I:

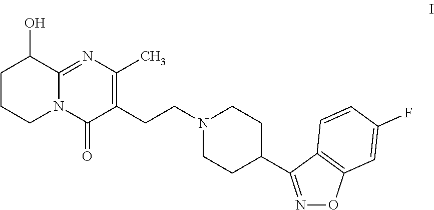

Paliperidone (available as INVEGA®) is an atypical antipsychotic developed by Janssen Pharmaceutica and its first synthesis was disclosed in U.S. Pat. No. 5,158,952.

Processes for the preparation of paliperidone and related compounds are disclosed in U.S. Pat. Nos. 5,158,952; 5,254,556; 5,688,799 and 6,320,048.

According to U.S. Pat. No. 5,158,952 (hereinafter referred to as the '952 patent), paliperidone is prepared by the reaction of 3-(2-chloroethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole in the presence of a base in a reaction inert solvent, and optionally in the presence of a phase transfer catalyst.

The reaction-inert solvents include water; an aromatic solvent, e.g., benzene, methylbenzene, dimethylbenzene, chlorobenzene, methoxybenzene and the like; a C₁₋₆ alkanol, e.g., methanol, ethanol, 1-butanol and the like; a ketone, e.g., 2-propanone, 4-methyl-2-pentanone and the like; an ester, e.g., ethyl acetate, γ-butyrolactone and the like; an ether, e.g., 1,1′-oxybisethane, tetrahydrofuran, 1,4-dioxane and the like; a dipolar aprotic solvent, e.g., N,N-dimethylformamide, N,N-dimethylacetamide, dimethylsulfoxide, pyridine, 1,3-dimethyl-3,4,5,6-tetrahydro-2(1H)-pyrimidinone, 1,3-dimethyl-2-imidazolidinone, 1,1,3,3-tetramethylurea, 1-methyl-2-pyrrolidinone, nitrobenzene, acetonitrile and the like; or a mixture thereof. The bases include inorganic bases such as, for example, an alkali metal or an earth alkaline metal carbonate, hydrogen carbonate, hydroxide, oxide, carboxylate, alkoxide, hydride or amide, e.g., sodium carbonate, sodium hydrogen carbonate, potassium carbonate, sodium hydroxide, calcium oxide, sodium acetate, sodium methoxide, sodium hydride, sodium amide and the like, or an organic base such as, for example, a tertiary amine, e.g., N,N-diethylethanamine, N-(1-methylethyl)-2-propanamine, 4-ethylmorpholine, 1,4-diazabicyclo[2.2.2]octane, pyridine and the like. The phase transfer catalysts include trialkylphenylmethylammonium, tetraalkylammonium, tetraalkylphosphonium, tetraarylphosphonium halide, hydroxide, hydrogen sulfate, and the like.

The reaction mixture containing paliperidone obtained according to the method of the '952 patent is then subjected to evaporation and the oily residue is extracted with trichloromethane followed by water washings. The organic layer is dried, filtered and evaporated followed by column chromatographic purifications over silica gel using a mixture of trichloromethane and methanol. The pure fractions are collected and the eluent is evaporated. The resulting residue is crystallized from 2-propanone. After cooling, the precipitated product is filtered off, washed with a mixture of 2-propanol and 2,2′-oxybispropane, and recrystallized from 2-propanol to produce paliperidone.

Paliperidone obtained by the process described in the '952 patent does not have satisfactory purity for pharmaceutical use. Unacceptable amounts of impurities are generally formed along with paliperidone. In addition, the process involves the additional step of column chromatographic purifications. Methods involving column chromatographic purifications are generally undesirable for large-scale operations, thereby making the process commercially unfeasible.

According to U.S. Pat. Nos. 5,158,952 and 5,254,556 (hereinafter referred to as the '556 patent), the key intermediate, 3-(2-chloroethyl)-6,7,8,9-tetrahydro-9-hydroxy-2-methyl-4H-pyrido[1,2-a]-pyrimidin-4-one, is prepared by the reaction of an optionally protected 2-aminopyridine derivative with an α-acyl lactone compound in the presence of an activating reagent and in a suitable reaction-inert solvent such as toluene at a temperature 90° C., followed by treatment with ammonium hydroxide to produce a protected or unprotected 9-hydroxy-3-(2-chloroethyl)-2-methyl-4H-pyrido[1,2-a]pyrimidin-4-one compound. The activating reagents include halogenating reagents such as, for example, phosphoryl chloride, phosphoryl bromide, phosphorous trichloride, thionyl chloride, and preferably phosphoryl chloride. The reaction mass is extracted with the solvents such as trichloromethane and then subjected to column chromatographic purifications. The 9-hydroxy-3-(2-chloroethyl)-2-methyl-4H-pyrido[1,2-a]pyrimidin-4-one compound is further catalytically hydrogenated in a suitable reaction-inert solvent in the presence of hydrogen, optionally at an elevated temperature and/or pressure, with a catalyst such as, for example, palladium on charcoal and the like, to produce 3-(2-chloroethyl)-6,7,8,9-tetrahydro-9-hydroxy-2-methyl-4H-pyrido[1,2-a]-pyrimidin-4-one. The reaction-inert solvents suitable for said catalytic hydrogenation reaction comprise water; C₁ alkanols, e.g., methanol, ethanol, 2-propanol and the like; ethers, e.g., 1,1′-oxybisethane, 1,4-dioxane, tetrahydrofuran, 2-methoxyethanol and the like; halogenated hydrocarbons, e.g., trichloromethane and the like; dipolar aprotic solvents, e.g., N,N-dimethylformamide and the like; esters, e.g., ethyl acetate, butyl acetate and the like; or a mixture of such solvents.

The intermediate compound, 3-(2-chloroethyl)-6,7,8,9-tetrahydro-9-hydroxy-2-methyl-4H-pyrido[1,2-a]-pyrimidin-4-one, obtained by the process described in the '952 and the '556 patents does not have satisfactory purity. Unacceptable amounts of impurities are generally formed during the reaction between the 2-aminopyridine compound and the α-acyl lactone compound. The process also suffers from disadvantages since the processes involve tedious and cumbersome procedures such as the use of additional solvents like toluene, higher temperatures, and column chromatographic purifications, thus resulting in low overall yields of the product. Methods involving column chromatographic purifications are generally undesirable for large-scale operations, thereby making the process commercially unfeasible.

According to U.S. Pat. No. 5,688,799 (hereinafter referred to as the '799 patent), the 9-hydroxy-3-(2-chloroethyl)-2-methyl-4H-pyrido[1,2-a]pyrimidin-4-one is prepared by the reaction of 2-amino-3-hydroxypyridine with 2-acetylbutyrolactone in the presence of p-toluenesulfonic acid in xylene solvent at reflux temperature overnight using a water separator to yield 9-hydroxy-3-(2-hydroxyethyl)-2-methyl-4H-pyrido[1,2-a]pyrimidin-4-one. The 9-hydroxy-3-(2-hydroxyethyl)-2-methyl-4H-pyrido[1,2-a]pyrimidin-4-one is converted into its hydrochloride salt, followed by reaction with thionyl chloride in dimethylformamide to produce 9-hydroxy-3-(2-chloroethyl)-2-methyl-4H-pyrido[1,2-a]pyrimidin-4-one.

The synthetic route described in the '799 patent involves a lengthy process, the yields obtained in this process are very low, and also the process produces a product of unsatisfactory purity. This process is also commercially unfeasible. Moreover, the intermediate compound, 9-hydroxy-3-(2-hydroxyethyl)-2-methyl-4H-pyrido[1,2-a]pyrimidin-4-one, obtained in this process is poorly soluble in xylene, resulting in deposit formation on the wall of the reaction vessel and discoloration of the reaction mixture to black. Furthermore, the reaction with thionyl chloride is characterized by a very strong smell, likely caused by reaction of residual 2-acetylbutyrolactone remaining from the previous step. The process generally results in a product of unreproducible yield and quality.

PCT Patent Publication No. WO 2006/027370 (hereinafter referred to as the '370 application) describes a modified process for preparation of 9-hydroxy-3-(2-hydroxyethyl)-2-methyl-4H-pyrido[1,2-a]pyrimidin-4-one involving the reaction of 2-amino-3-hydroxypyridine with 2-acetylbutyrolactone in the presence of p-toluenesulfonic acid and using chlorobenzene as a solvent at reflux temperature, i.e., at 125° C. The product is isolated by a process involving the addition of alcoholic solvent and filtration of the reaction mixture at 90-95° C.

The process described in the '370 application involves a lengthy process, the reaction proceeds at a high temperature i.e., above 125° C., it takes 19 hours for reaction completion, and also involves the hazard of filtration of a reaction mixture containing flammable solvents at 90-95° C. which poses problems in scale up operations. Based on the aforementioned drawbacks, this process may be unsuitable for preparation of the 9-hydroxy-3-(2-hydroxyethyl)-2-methyl-4H-pyrido[1,2-a]pyrimidin-4-one at laboratory scale and commercial scale operations.

PCT Publication No. WO 2008/087557 A2 (hereinafter referred to as the '557 application) discloses an improved process for the preparation of paliperidone intermediate, 9-hydroxy-3-(2-chloroethyl)-2-methyl-4H-pyrido[1,2-a]pyrimidin-4-one or its hydrochloride salt thereof, comprising: a) reacting 2-amino-3-hydroxypyridine with 2-acetylbutyrolactone in the presence of phosphorous oxychloride to produce a reaction mass; b) quenching the reaction mass in a mixture of ice and water to form a quenched reaction mass; c) adjusting the pH of the quenched reaction mass to 4-6 with a base to produce a separated 9-hydroxy-3-(2-chloroethyl)-2-methyl-4H-pyrido[1,2-a]pyrimidin-4-one; d) collecting the separated 9-hydroxy-3-(2-chloroethyl)-2-methyl-4H-pyrido[1,2-a]pyrimidin-4-one; and e) optionally converting the 9-hydroxy-3-(2-chloroethyl)-2-methyl-4H-pyrido[1,2-a]pyrimidin-4-one into its hydrochloride salt by reacting the separated compound with an alcoholic or gaseous hydrogen chloride in a solvent selected from an alcoholic solvent and an aromatic solvent.

The process described in the '557 application also suffers from disadvantages since the yields of the paliperidone intermediate, 9-hydroxy-3-(2-chloroethyl)-2-methyl-4H-pyrido[1,2-a]pyrimidin-4-one or its hydrochloride salt, obtained are very low (i.e., 29 to 32% only), thereby making the process commercially unfeasible.

PCT Publication No. WO 2008/021342 A2 (hereinafter referred to as the '342 application) discloses six crystalline forms of paliperidone (Forms I-VI), characterizes them by powder X-ray diffraction (P-XRD) and solid state ¹³C NMR, and processes for their preparation thereof.

PCT publication No. WO 2008/021345 discloses a process for preparing paliperidone comprising reacting 3-(2-chloro ethyl)-6,7,8,9-tetrahydro-9-hydroxy-2-methyl-4H-pyrido[1,2-a]-pyrimidin-4-one or a salt thereof with 6-fluoro-3-piperidino-1,2-benzisoxazol or a salt thereof in a solvent in the presence of an inorganic base, optionally in the presence of metal iodide and phase transfer catalyst. This process produces paliperidone with 90% purity, which further requires multiple purifications/crystallizations to provide high purity paliperidone, thus resulting in low overall yields of the product.

PCT Publication No. WO 2008/021346 (hereinafter referred to as the '346 application) teaches four general purification methods of paliperidone. According to the first purification process, paliperidone is crystallized from a solvent selected from the group consisting of: a C₃₋₆ ketone or a mixture thereof with water, N-methylpyrrolidone, C₃₋₆ amides, halo-substituted C₆₋₁₂ aromatic hydrocarbons, propylene glycol, dimethyl sulfoxide, di-methyl carbonate, C₁₋₄ alkyl alcohols, a mixture of a C₁₋₈ alkyl alcohol and water, acetonitrile or a mixture thereof with water, C₂₋₆ alkyl acetates or their mixture with water, cellosolve, dimethyl carbonate, polyethylene glycol methyl ether and C₂₋₈ ethers. The crystallization involves dissolving paliperidone in the above solvents, heating the reaction mixture to allow complete dissolution, followed by cooling of the obtained solution, whereby paliperidone crystallizes. The second purification process of the '346 application comprises crystallizing paliperidone by combining a solution of paliperidone in a first solvent, selected from the group consisting of: dichloromethane, dioxane and C₁₋₄ alkyl alcohols, with an anti-solvent selected from the group consisting of C₃₋₆ ketones, C₃₋₆ ethers, acetonitrile, C₃₋₇ straight and cyclic carbohydrates, C₆₋₁₂ aromatic carbohydrates and water. The third purification process of the '346 application comprises slurrying paliperidone in an organic solvent selected from C₁₋₄ alkyl alcohols, C₃₋₅ ketones and water. The fourth purification process of the '346 application comprises dissolving paliperidone in a mixture of acetone and water, admixing the solution with finely powdered carbon, and filtering the admixture to obtain pure paliperidone.

Two impurities of paliperidone have been disclosed in the '346 application and these impurities are characterized as 3-[2-[4-(6-fluorobenzo[d]isoxazol-3-yl)-1-oxypiperidin-1-yl]ethyl]-7-hydroxy-4-methyl-1,5-diazabicyclo[4.4.0]deca-3,5-dien-2-one (PLP-NO) and 2-[4-(6-fluoro-1,2-benzisoxazol-3-yl)piperidin-1-carboxylic acid]-7-hydroxy-2-methyl-6,7,8,9-tetra-hydro-4H-pyrido[1,2-a]pyrimidin-4-one-3-yl-ethyl ester (PLP-car), having the following structural formulae:

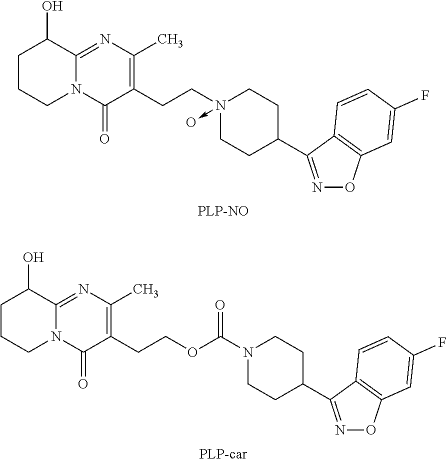

The '346 application suffers from drawbacks since the purification procedures described in this application reduce the content of the PLP-NO impurity in paliperidone from about 0.67% to about 0.35% and from about 0.41% to about 0.20%, which further requires repetition of purification processes to further reduce the content of impurities in order to provide high purity paliperidone, thus resulting in low yields of the product. Therefore, the purification processes described in '346 application are not suitable for preparation of pharmaceutically acceptable grade paliperidone.

U.S. Patent Application No. 20080281100 (hereinafter referred to as the '100 application) discloses a potential impurity of paliperidone referred to as “impurity X” having a relative retention time (“RRT”) of about 1.27, as relative to the retention time of paliperidone as measured by HPLC, and processes for purifying paliperidone contaminated with impurity X. As per the purification processes exemplified in the '100 application, it has been observed that the content of impurity in the paliperidone obtained after purification is more than 0.15%, which is not acceptable as per ICH guidelines. Therefore, the purification processes described in the '100 application are not suitable for preparation of pharmaceutically acceptable grade paliperidone.

PCT publication No. WO 2009/118655 (hereinafter referred to as the '655 application) discloses a potential impurity of paliperidone characterized as 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione (‘paliperidone keto impurity’) having a relative retention time (“RRT”) of about 0.96, as relative to the retention time of paliperidone as measured by HPLC, and processes for purifying paliperidone contaminated with keto impurity. The paliperidone keto impurity (designated herein as the ‘9-keto impurity’ of formula C) has the following structural formula:

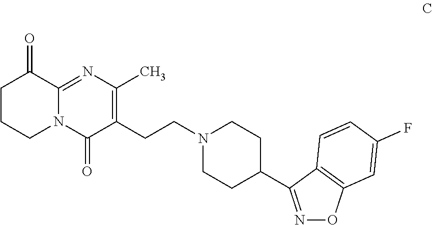

The '655 application further teaches that paliperidone prepared by the prior art procedures contains about above 1 wt % and up to 3 wt % of the keto impurity and the content of the keto impurity could be further reduced to about 0.5 wt % by using the re-crystallization procedures described in the prior art. However, the amount of keto impurity could not be reduced to below 0.2 wt % or eliminated completely using the prior art purification procedures.

According to the '655 application, the purification process, for preparing highly pure paliperidone or a pharmaceutically acceptable salt thereof substantially free of keto impurity, comprises the steps of: a) providing a first solution or a suspension of crude paliperidone in a first organic solvent selected from the group consisting of alcohols, amides, organosulfur solvents, and mixtures thereof; b) isolating or recovering the paliperidone as a solid from the solution or suspension from step-(a); c) dissolving the solid from (b) in a second organic solvent to form a second solution, wherein the second organic solvent is a C₁ to C₆ straight or branched chain alcohol; d) combining the second solution obtained in step-(c) with a reducing agent to produce a reaction mass, wherein the reducing agent is a metal hydride; e) optionally, filtering the reaction mass obtained in step-(d) to remove extraneous matter; and f) isolating highly pure paliperidone substantially free of keto impurity from the reaction mass, and optionally converting the highly pure paliperidone obtained into a pharmaceutically acceptable salt.

The purification process described in the '655 application also suffers from disadvantages since the yields of the pure paliperidone or a pharmaceutically acceptable salt obtained after purification process are very low.

PCT publication No. WO 2009/010988 (hereinafter referred to as the '988 application) describes a method for purification of paliperidone comprising dissolving paliperidone in aqueous mineral acid such as hydrochloric acid, adjusting the pH to 3.5-6.5 using ammonia or sodium bicarbonate, isolating pure paliperidone acid addition salt by filtration, suspending the salt in water, adjusting the pH of suspension to 8.5-9.0 with potassium carbonate or ammonia, filtration of resultant solid and drying after washing with an alcoholic solvent.

The process described in the '988 application also suffers from disadvantage since the reported overall yield of the pure paliperidone or a pharmaceutically acceptable salt obtained after purification process is very low (i.e. 34.74% only), thereby making the process commercially unfeasible. Furthermore, it has been observed that the content of 9-keto impurity in the paliperidone obtained after purification, as per the purification processes exemplified in the '988 application, is more than 0.15%, which is not acceptable as per ICH guidelines. Therefore, the purification processes described in the '988 application are not suitable for preparation of pharmaceutically acceptable grade paliperidone.

PCT Publication No. WO 2009/060297 describes certain acid addition salts of paliperidone derived from an acid selected from hydrochloric acid, hydrobromic acid, hydroiodic acid, ortho phosphoric acid, fumaric acid or oxalic acid. The '297 publication further discloses crystalline forms of paliperidone hydrochloride, paliperidone hydrobromide, paliperidone phosphate and paliperidone fumarate, and characterizes them by powder X-ray diffraction.

However, the disadvantage of the prior art purification processes is that the yields of the product (paliperidone or a pharmaceutically acceptable salt) are drastically reduced during the purification processes to prepare pharmaceutically acceptable grade paliperidone. Moreover, most of the prior art processes also suffer from the same drawback since they could not reduce or eliminate the content of 9-keto impurity, which is a persistent impurity in the paliperidone, to less than 0.15% (which is not acceptable as per ICH guidelines), even after carrying out the extensive and lengthy purification processes.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in paliperidone or any active pharmaceutical ingredient (API) are undesirable and might be harmful.

Regulatory authorities worldwide require that drug manufactures isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards have been set with the intention of ensuring that an API is as free of impurities as possible, and, thus, are as safe as possible for clinical use. The United States Food and Drug Administration guidelines recommend that the amounts of some impurities limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRt”) to identify impurities. The RRt of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

Thus, there is a need for highly efficient processes for preparing pure paliperidone or a pharmaceutically acceptable salt thereof substantially free of impurities with higher yields.

### SUMMARY

In one aspect, provided herein is an isolated methanoyl compound, 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one, having the following structural formula A:

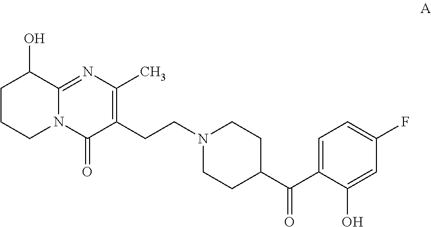

In another aspect, provided herein is an impurity of paliperidone, paliperidone methanoyl impurity, 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one, of formula A.

In another aspect, encompassed herein is a process for synthesizing and isolating the methanoyl compound of formula A, also referred to as the “methanoyl impurity”.

In another aspect, provided herein is an isolated dehydroxy compound, 3-[2-[4-(6-fluoro-1,2-benzisoxazol-3-yl)-1-piperidinyl]ethyl]-2-methyl-4H-pyrido[1,2-a]pyrimidin-4-one, having the following structural formula B:

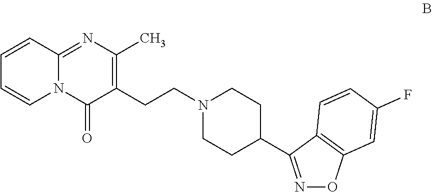

In another aspect, provided herein is an impurity of paliperidone, dehydroxy impurity, 3-[2-[4-(6-fluoro-1,2-benzisoxazol-3-yl)-1-piperidinyl]ethyl]-2-methyl-4H-pyrido[1,2-a]pyrimidin-4-one, of formula B.

In another aspect, encompassed herein is a process for synthesizing and isolating the dehydroxy compound of formula B, also referred to as the “dehydroxy impurity”.

In another aspect, encompassed herein is a process for synthesizing and isolating paliperidone 9-keto impurity, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione, of formula C:

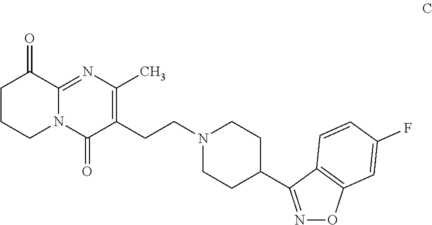

In another aspect, provided herein is a highly pure paliperidone or a pharmaceutically acceptable salt thereof substantially free of methanoyl, dehydroxy and 9-keto impurities.

In still further aspect, encompassed herein is a process for preparing the highly pure paliperidone or a pharmaceutically acceptable salt thereof substantially free of methanoyl, dehydroxy and 9-keto impurities.

In another aspect, provided herein is a pharmaceutical composition comprising highly pure paliperidone or a pharmaceutically acceptable salt thereof substantially free of methanoyl, dehydroxy and 9-keto impurities, and one or more pharmaceutically acceptable excipients.

In still another aspect, provided herein is a pharmaceutical composition comprising highly pure paliperidone or a pharmaceutically acceptable salt thereof substantially free of methanoyl, dehydroxy and 9-keto impurities made by the process disclosed herein, and one or more pharmaceutically acceptable excipients.

In still further aspect, encompassed is a process for preparing a pharmaceutical formulation comprising combining highly pure paliperidone or a pharmaceutically acceptable salt thereof substantially free of methanoyl, dehydroxy and 9-keto impurities with one or more pharmaceutically acceptable excipients.

Preferable pharmaceutically acceptable salts of paliperidone include, but are not limited to, hydrochloride, hydrobromide, oxalate, nitrate, sulphate, phosphate, fumarate, succinate, maleate, fumarate, besylate, tosylate, tartrate, palmitate; and more preferably hydrochloride.

In another aspect, the highly pure paliperidone or a pharmaceutically acceptable salt thereof substantially free of methanoyl, dehydroxy and 9-keto impurities disclosed herein for use in the pharmaceutical compositions has a D₉₀ particle size of less than or equal to about 400 microns, specifically about 1 micron to about 300 microns, and most specifically about 10 microns to about 150 microns.

### DETAILED DESCRIPTION

According to one aspect, there is provided an isolated methanoyl compound, 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one, having the following structural formula A:

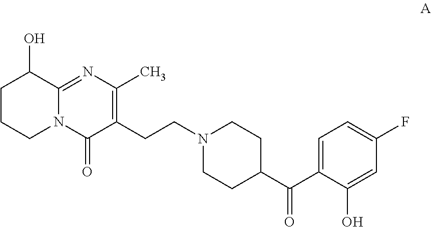

According to another aspect, there is provided an impurity of paliperidone, paliperidone methanoyl impurity, 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one, of formula A.

The paliperidone methanoyl impurity has been identified, isolated and synthesized. The methanoyl impurity was detected and resolved from paliperidone by HPLC with an RRT of 1.05. The structure of the compound of formula A was deduced with the aid of ¹H, ¹³C NMR and IR spectroscopy and FAB mass spectrometry. The parent ion at 429.48 is consistent with the assigned structure.

The present inventors have found that the methanoyl compound of formula A is formed as an impurity, in an amount of about 0.16% to about 0.5% as measured by HPLC, during the synthesis of paliperidone.

In one embodiment, the paliperidone methanoyl compound of formula A is prepared as per the process exemplified in the Example 9 as disclosed herein.

According to another aspect, there is provided an isolated dehydroxy compound, 3-[2-[4-(6-fluoro-1,2-benzisoxazol-3-yl)-1-piperidinyl]ethyl]-2-methyl-4H-pyrido[1,2-a]pyrimidin-4-one, having the following structural formula B:

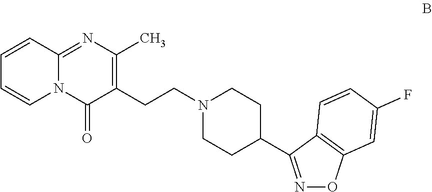

According to another aspect, there is provided an impurity of paliperidone, paliperidone dehydroxy impurity, 3-[2-[4-(6-fluoro-1,2-benzisoxazol-3-yl)-1-piperidinyl]ethyl]-2-methyl-4H-pyrido[1,2-a]pyrimidin-4-one, of formula B.

The paliperidone dehydroxy impurity has been identified, isolated and synthesized. The dehydroxy impurity was detected and resolved from paliperidone by HPLC with an RRT of 1.18. The structure of the compound of formula B was deduced with the aid of ¹H, ¹³C NMR and IR spectroscopy and FAB mass spectrometry. The parent ion at 406.45 is consistent with the assigned structure.

In another embodiment, the paliperidone dehydroxy compound of formula B is prepared as per the process exemplified in the Example 10 as disclosed herein.

According to another aspect, there is provided an isolated paliperidone 9-keto impurity, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione, having the following structural formula C:

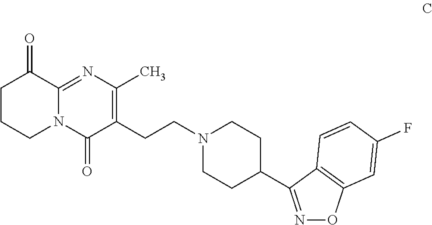

The paliperidone 9-keto impurity has been identified, isolated and synthesized. The 9-keto impurity was detected and resolved from paliperidone by HPLC with an RRT of 0.96. The structure of the compound of formula C was deduced with the aid of ¹H, ¹³C NMR and IR spectroscopy and FAB mass spectrometry. The parent ion at 424.46 is consistent with the assigned structure.

In another embodiment, the paliperidone 9-keto impurity of formula C is prepared as per the process exemplified in the Example 11 as disclosed herein.

Regarding the specific RRt values of impurities disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.02.

Thus there is a need for a method for determining the level of impurities in paliperidone samples and removing the impurities.

Extensive research and experimentation was carried out by the present inventors to reduce the level of the methanoyl, dehydroxy and 9-keto impurities in paliperidone. As a result, it has been found that the contents of methanoyl, dehydroxy and 9-keto impurities, formed in the synthesis of the paliperidone, can be reduced or substantially removed by the purification process disclosed herein.

According to another aspect, there is provided a highly pure paliperidone or a pharmaceutically acceptable salt thereof substantially free of methanoyl, dehydroxy and 9-keto impurities.

As used herein, “highly pure paliperidone or a pharmaceutically acceptable salt thereof substantially free of methanoyl, dehydroxy and 9-keto impurities” refers to paliperidone or a pharmaceutically acceptable salt thereof comprising the methanoyl, dehydroxy and 9-keto impurities, each one, in an amount of less than about 0.1 area-% as measured by HPLC. Specifically, the paliperidone, as disclosed herein, contains the methanoyl, dehydroxy and 9-keto impurities, each one, in an amount of less than about 0.07 area-%, more specifically less than about 0.05 area-%, still more specifically less than about 0.02 area-%, and most specifically is essentially free of one, or more, of the methanoyl, dehydroxy and 9-keto impurities.

In one embodiment, the highly pure paliperidone or a pharmaceutically acceptable salt thereof disclosed herein comprises one, or more, of the methanoyl, dehydroxy and 9-keto impurities, each one, in an amount of about 0.01 area-% to about 0.1 area-%, specifically in an amount of about 0.01 area-% to about 0.05 area-%, as measured by HPLC.

In another embodiment, the highly pure paliperidone or a pharmaceutically acceptable salt thereof disclosed herein has a total purity of greater than about 99.5%, specifically greater than about 99.8%, more specifically greater than about 99.9%, and most specifically greater than about 99.95% as measured by HPLC. For example, the purity of the highly pure paliperidone or a pharmaceutically acceptable salt thereof is about 99.5% to about 99.9%, or about 99.8% to about 99.99%.

In another embodiment, the highly pure paliperidone or a pharmaceutically acceptable salt thereof disclosed herein is essentially free of at least one, or more, of the methanoyl, dehydroxy and 9-keto impurities.

The term “paliperidone or a pharmaceutically acceptable salt thereof essentially free of at least one, or more, of the methanoyl, dehydroxy and 9-keto impurities” refers to paliperidone or a pharmaceutically acceptable salt thereof contains a non-detectable amount of one, or more, of the methanoyl, dehydroxy and 9-keto impurities as measured by HPLC.

Exemplary pharmaceutically acceptable salts of paliperidone include, but are not limited to, hydrochloride, hydrobromide, oxalate, nitrate, sulfate, phosphate, fumarate, succinate, maleate, besylate, tosylate, tartrate, palmitate; and more specifically hydrochloride.

According to another aspect, there is provided a process for preparing highly pure paliperidone or a pharmaceutically acceptable salt thereof substantially free of the methanoyl, dehydroxy and 9-keto impurities, comprising:
a) providing a first solution or suspension of crude paliperidone free base in a solvent;
b) adjusting the pH of the first solution or suspension obtained in step-(a) to below 3.5 with hydrochloric acid to produce a second solution or suspension;
c) basifying the second solution or suspension obtained in step-(b) with a base by adjusting the pH to about 5 to 6 to produce a third solution or suspension;
d) combining the third solution or suspension with a reducing agent selected from the group consisting of sodium dithionite, sodium dithionate, sodium metabisulfite and potassium metabisulfite to produce a fourth solution;
e) optionally, subjecting the fourth solution to carbon treatment or silica gel treatment;
f) adjusting the pH of the fourth solution to about 8.5 to 9 with a base to produce a slurry containing paliperidone free base;
g) recovering paliperidone free base as a solid from the slurry obtained in step-(f);
h) combining the paliperidone free base solid obtained in step-(g) with water to form an aqueous slurry; and
i) isolating and/or recovering the highly pure paliperidone substantially free of the impurities from the aqueous slurry; and optionally converting the highly pure paliperidone obtained into a pharmaceutically acceptable salt thereof.

Exemplary solvents used in step-(a) include, but are not limited to, water, an alcohol, a ketone, and mixtures thereof. The term solvent also includes mixtures of solvents.

In one embodiment, the solvent is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, and mixtures thereof.

Specifically, the solvent is selected from the group consisting of water, methanol, isopropyl alcohol, acetone, and mixtures thereof; and more specifically a mixture of water and methanol or isopropyl alcohol.

Step-(a) of providing a first solution of crude paliperidone free base includes dissolving paliperidone free base in the first solvent, or obtaining an existing solution from a previous processing step.

In one embodiment, the crude paliperidone is dissolved in the first solvent at a temperature of about 0° C. to the reflux temperature of the solvent used, specifically at about 25° C. to about 100° C., and more specifically at about 40° C. to about 80° C.

As used herein, “reflux temperature” means the temperature at which the solvent or solvent system refluxes or boils at atmospheric pressure.

In another embodiment, step-(a) of providing a suspension of crude paliperidone free base includes suspending crude paliperidone free base in the first solvent while stirring at a temperature of about 0° C. to the reflux temperature of the solvent used. In one embodiment, the suspension is stirred at a temperature of about 25° C. to about 100° C. for at least 15 minutes and more specifically at a temperature of about 40° C. to about 80° C. for about 30 minutes to about 10 hours.

In another embodiment, the solution or suspension in step-(a) is prepared by reacting 3-(2-chloroethyl)-6,7,8,9-tetrahydro-9-hydroxy-2-methyl-4H-pyrido[1,2-a]-pyrimidin-4-one or an acid addition salt thereof with 6-fluoro-3-(4-piperidinyl)-1,2-benzisoxazole or an acid addition salt thereof in the presence of a base, optionally in the presence of a phase transfer catalyst, in a reaction inert solvent under suitable conditions to produce a reaction mass containing crude paliperidone free base, followed by usual work up such as washings, extractions, evaporations, filtrations, etc. In one embodiment, the work-up includes dissolving, suspending or extracting the resulting crude paliperidone in the first solvent at a temperature of about 0° C. to the reflux temperature of the solvent used, specifically at about 25° C. to about 100° C., and more specifically at about 40° C. to about 80° C.

Exemplary phase transfer catalysts suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole include, but are not limited to, quaternary ammonium salts substituted with a group such as a straight or branched alkyl group having 1 to about 18 carbon atoms, a phenyl lower alkyl group including a straight or branched alkyl group having 1 to 6 carbon atoms which is substituted by an aryl group and phenyl group, e.g., tetrabutylammonium chloride, tetrabutylammonium bromide, tetrabutylammonium fluoride, tetrabutylammonium iodide, tetrabutylammonium hydroxide, tetrabutylammonium hydrogen sulfate, tributylmethylammonium chloride, tributylbenzylammonium chloride, tetraethylammonium chloride, tetramethylammonium chloride, tetrapentylammonium chloride, tetrapentylammonium bromide, tetrahexyl ammonium chloride, benzyldimethyloctylammonium chloride, methyltrihexylammonium chloride, benzylmethyloctadecanylammonium chloride, methyltridecanylammonium chloride, benzyltripropylammonium chloride, benzyltriethyl ammonium chloride, phenyltriethylammonium chloride and the like; phosphonium salts substituted with a residue such as a straight or branched alkyl group having 1 to about 18 carbon atoms, e.g., tetrabutylphosphonium chloride and the like; and pyridinium salts substituted with a straight or branched alkyl group having 1 to about 18 carbon atoms, e.g., 1-dodecanylpyridinium chloride and the like.

Specific phase transfer catalysts are tetrabutylammonium bromide, tetrabutylphosphonium bromide, tetrabutylammonium chloride, tetrabutylphosphonium chloride, benzyltriethylammonium chloride, tetrabutylammonium hydrogen sulfate, and more specifically tetrabutylammonium bromide.

Exemplary reaction inert solvents suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole include, but are not limited to, water, alcohols, ketones, cyclic ethers, aliphatic ethers, hydrocarbons, chlorinated hydrocarbons, nitriles, esters, polar aprotic solvents, and the like, and mixtures thereof. In one embodiment, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof.

In one embodiment, the base suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole is an organic or inorganic base. Specific organic bases are triethyl amine, trimethylamine, N,N-diisopropylethylamine, N-methylmorpholine and N-methylpiperidine.

In another embodiment, the base is an inorganic base. Exemplary inorganic bases include, but are not limited to, ammonia; hydroxides, alkoxides, carbonates and bicarbonates of alkali or alkaline earth metals. Specific inorganic bases are ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically ammonia, sodium hydroxide, potassium hydroxide, sodium carbonate and potassium carbonate.

Alternatively, the solution or suspension in step-(a) is prepared by treating an acid addition salt of crude paliperidone with a base to liberate paliperidone free base followed by extracting, dissolving or suspending the crude paliperidone in the first solvent at a temperature of about 0° C. to the reflux temperature of the solvent used, specifically at about 25° C. to about 100° C., and more specifically at about 40° C. to about 80° C.

In another embodiment, the acid addition salt of paliperidone is derived from a therapeutically acceptable acid such as hydrochloric acid, acetic acid, propionic acid, sulfuric acid, nitric acid, succinic acid, maleic acid, fumaric acid, citric acid, glutaric acid, citraconic acid, glutaconic acid, tartaric acid, malic acid, and ascorbic acid. A specific salt is paliperidone hydrochloride.

The treatment of an acid addition salt with a base is carried out in a solvent and the selection of solvent is not critical. A wide variety of solvents such as chlorinated solvents, alcohols, ketones, hydrocarbon solvents, esters, ether solvents etc., can be used.

The base used herein can be selected from the group of inorganic and organic bases as described above.

The first solution or suspension obtained in step-(a) is optionally stirred at a temperature of about 25° C. to the reflux temperature of the solvent used for at least 15 minutes, and specifically at a temperature of about 40° C. to the reflux temperature of the solvent used for about 20 minutes to about 8 hours.

In one embodiment, the pH of the first solution or suspension in step-(b) is adjusted between about 3 and 3.5.

In another embodiment, the hydrochloric acid used in step-(b) is in the form of concentrated hydrochloric acid or aqueous hydrochloric acid or in the form of hydrogen chloride gas or hydrogen chloride dissolved in an organic solvent selected from the group consisting of an alcohol and a ketone. In one embodiment, the organic solvent is selected from the group consisting of ethanol, methanol, isopropyl alcohol, and acetone.

In one embodiment, the pH of the second solution or suspension in step-(c) is adjusted between about 5.5 and 6.

The base used in step-(c) can be selected from the group of inorganic and organic bases as described above. Specifically, the base is an inorganic base and more specifically ammonia. In one embodiment, the ammonia used is in the form of aqueous ammonia or in the form of ammonia dissolved in an organic solvent selected from the group consisting of an alcohol and a ketone. In one embodiment, the organic solvent is selected from the group consisting of ethanol, methanol, isopropyl alcohol, and acetone.

The third solution or suspension obtained in step-(c) is optionally stirred at a temperature of about 25° C. to the reflux temperature of the solvent used for at least 15 minutes, and specifically at a temperature of about 40° C. to the reflux temperature of the solvent used for about 20 minutes to about 4 hours.

Combining of the third solution or suspension with the reducing agent in step-(d) is done in a suitable order, for example, the third solution or suspension is added to the reducing agent, or alternatively, the reducing agent is added to the third solution or suspension. The addition is, for example, carried out drop wise or in one portion or in more than one portion. The addition is specifically carried out at a temperature of about 30° C. to about 90° C. for at least 10 minutes and more specifically at a temperature of about 40° C. to about 80° C. for about 30 minutes to about 4 hours. After completion of addition process, the resulting mass is stirred at a temperature of below about 100° C. for at least 5 minutes and more specifically at about 50° C. to about 80° C. for about 10 minutes to about 10 hours.

In one embodiment, the reducing agent is used directly or in the form of an aqueous solution or in the form of reducing agent dissolved in an organic solvent, wherein the organic solvent is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, and mixtures thereof.

The carbon treatment or silica gel treatment in step-(e) is carried out by methods known in the art, for example, by stiffing the fourth solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate by removing charcoal or silica gel. Specifically, the finely powdered carbon is an active carbon. A specific mesh size of silica gel is 40-500 mesh, and more specifically 60-120 mesh.

The base used in step-(f) can be selected from the group of inorganic and organic bases as described above. Specifically, the base is an inorganic base and more specifically ammonia. In one embodiment, the ammonia used is in the form of aqueous ammonia or in the form of ammonia dissolved in an organic solvent as described above.

The slurry obtained in step-(f) is optionally stirred at a temperature of about 15° C. to the reflux temperature of the solvent used for at least 15 minutes, and specifically at a temperature of about 20° C. to about 35° C. for about 20 minutes to about 4 hours.

The recovery of paliperidone free base in step-(g) is accomplished by techniques such as filtration, filtration under vacuum, decantation, centrifugation, or a combination thereof.

In one embodiment, the paliperidone free base is recovered by filtration employing a filtration media of, for example, a silica gel or celite.

Combining of the paliperidone solid with water in step-(h) is done in a suitable order as described above, for example, the paliperidone solid is added to water, or alternatively, water is added to the paliperidone solid. The addition is specifically carried out at a temperature of below about 100° C. and more specifically at a temperature of about 30° C. to about 80° C. After completion of the addition process, the resulting mass is stirred at a temperature of below about 100° C. for at least 15 minutes and more specifically at about 30° C. to about 80° C. for about 20 minutes to about 10 hours.

The isolation of highly pure paliperidone base in step-(i) is carried out, for example, by cooling the aqueous slurry while stirring at a temperature of below 30° C. for at least 15 minutes, specifically at about 0° C. to about 30° C. for about 30 minutes to about 20 hours, and more specifically at about 20° C. to about 30° C. for about 1 hour to about 10 hours.

The recovery of highly pure paliperidone base in step-(i) is accomplished by techniques such as filtration, filtration under vacuum, decantation, centrifugation, or a combination thereof. In one embodiment, the paliperidone base is recovered by filtration employing a filtration media of, for example, a silica gel or celite.

In one embodiment, the highly pure paliperidone base obtained in step-(i) is optionally suction dried followed by suspending in an organic solvent, stiffing the suspension at a temperature of about 30° C. to the reflux temperature of the organic solvent used and then recovering the pure paliperidone by the methods as described above. Specifically, the organic solvent is selected from the group consisting of an alcohol, a ketone, and mixtures thereof; and more specifically, methanol, isopropyl alcohol, acetone, and mixtures thereof.

Pharmaceutically acceptable salts of paliperidone can be prepared in high purity by using the highly pure paliperidone substantially free of impurities obtained by the method disclosed herein, by known methods.

Specific pharmaceutically acceptable salts of paliperidone are hydrochloride, hydrobromide, oxalate, nitrate, sulphate, phosphate, fumarate, succinate, maleate, besylate, tosylate, palmitate and tartrate; and more specifically hydrochloride.

The highly pure paliperidone or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

According to another aspect, there is provided a process for the preparing pure paliperidone, comprising:
a) reacting 3-(2-chloroethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole hydrochloride in the presence of a base, optionally in the presence of a metal iodide and a phase transfer catalyst, in a first solvent under atmospheric pressure or high pressure to produce a reaction mass containing crude paliperidone free base;
b) isolating crude paliperidone free base as a solid from the reaction mass obtained step-(a);
c) combining the crude paliperidone free base with water to form an aqueous slurry;
d) isolating paliperidone free base as a solid from the aqueous slurry;
e) dissolving the solid paliperidone free base obtained in step-(d) in a second solvent to produce a first solution;
f) combining the first solution with L-(+)-tartaric acid to produce a second solution or suspension;
g) isolating the solid state form of paliperidone L-(+)-tartrate salt from the second solution or suspension;
h) dissolving or suspending the paliperidone L-(+)-tartrate salt in a third solvent to produce a third solution or suspension;
i) combining the third solution or suspension with a reducing agent selected from the group consisting of sodium dithionite, sodium dithionate, sodium metabisulfite and potassium metabisulfite to produce a fourth solution or suspension;
j) adjusting the pH of the solution or suspension obtained in step-(h) or step-(i) to about 8 to 10 with a base to produce a fifth solution or suspension containing paliperidone free base; and
k) isolating and/or recovering the pure paliperidone free base from the fifth solution or suspension obtained in step-(j).

In one embodiment, the reaction in step-(a) is carried out under atmospheric pressure. In another embodiment, the reaction in step-(a) is carried out under high pressure.

As used herein the term “high pressure” refers to the pressure of about 2 to 15 kg/cm², specifically about 4 to 10 kg/cm², and more specifically about 5 to 8 kg/cm².

It has been observed that when the reaction in step-(a) is carried out under atmospheric pressure it takes about 40 hours to about 50 hours for reaction completion whereas the reaction is carried out under high pressure the reaction will be completed within 5 to 7 hours.

The base used in step-(a) is selected from the group of organic and inorganic bases as described above.

In one embodiment, the base is an organic base selected from the group consisting of triethyl amine, trimethylamine, N,N-diisopropylethylamine, N-methylmorpholine and N-methylpiperidine. A specific organic base is N,N-diisopropylethylamine.

In another embodiment, the base is an inorganic base selected from the group consisting of ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide. Specific inorganic bases are ammonia, sodium hydroxide, potassium hydroxide, sodium carbonate and potassium carbonate; and more specifically sodium carbonate.

Exemplary metal iodides employed in step-(a) include, but are not limited to, potassium iodide, sodium iodide, and the like.

The phase transfer catalyst employed in step-(a) is selected from the group as described above. Specific phase transfer catalysts are tetrabutylammonium bromide, tetrabutylphosphonium bromide, tetrabutylammonium chloride, tetrabutylphosphonium chloride, benzyltriethylammonium chloride, tetrabutylammonium hydrogen sulfate, and more specifically tetrabutylammonium bromide.

Exemplary first solvents used in step-(a) include, but are not limited to, an alcohol, a ketone, tetrahydrofuran, N,N-dimethylformamide, N,N-dimethylacetamide, dimethylsulfoxide, and mixtures thereof. The term solvent also includes mixtures of solvents.

Specifically, the first solvent is selected from the group consisting of methanol, ethanol, isopropyl alcohol, propanol, t-butanol, n-butanol, acetone, methyl ethyl ketone, methyl isobutyl ketone, diethyl ketone, tetrahydrofuran, N,N-dimethylformamide, N,N-dimethylacetamide, dimethylsulfoxide, and mixtures thereof; more specifically, the solvent is selected from the group consisting of methanol, ethanol, isopropyl alcohol, N,N-dimethylformamide, and mixtures thereof; and a most specific solvent is methanol.

In one embodiment, the reaction in step-(a) is carried out under atmospheric pressure at a temperature of about 45° C. to about 65° C. for at least 35 hours, and more specifically at about 55° C. to about 60° C. for about 40 hours to about 45 hours.

In another embodiment, the reaction in step-(a) is carried out under high pressure of about 4 to 10 kg/cm² at a temperature of about 60° C. to about 90° C. for at least 2 hours, and more specifically under pressure of about 5 to 8 kg/cm² at about 70° C. to about 80° C. for about 4 hours to about 7 hours.

The reaction mass obtained in step-(a) is subjected to usual work-up such as washings, extractions, layer separations, evaporations, acid/base treatments, filtrations, or a combination thereof.

The isolation in step-(b) is carried out, for example, by cooling the reaction mass while stiffing at a temperature of below 35° C. for at least 15 minutes, specifically at about 0° C. to about 30° C. for about 30 minutes to about 10 hours, and more specifically at about 20° C. to about 30° C. for about 1 hour to about 5 hours.

The solid obtained in step-(b) is recovered by techniques such as filtration, filtration under vacuum, decantation, centrifugation, or a combination thereof. In one embodiment, the solid is recovered by filtration employing a filtration media of, for example, a silica gel or celite.

Combining of the paliperidone solid with water in step-(c) is done in a suitable order as described above, for example, the paliperidone solid is added to water, or alternatively, water is added to the paliperidone solid. The addition is specifically carried out at a temperature of below about 100° C. and more specifically at a temperature of about 30° C. to about 80° C. After completion of addition process, the resulting mass is stirred at a temperature of below about 100° C. for at least 15 minutes and more specifically at about 30° C. to about 80° C. for about 20 minutes to about 10 hours.

The isolation in step-(d) is carried out, for example, by cooling the reaction mass while stiffing at a temperature of below 35° C. for at least 15 minutes, specifically at about 0° C. to about 30° C. for about 30 minutes to about 10 hours, and more specifically at about 20° C. to about 30° C. for about 1 hour to about 5 hours.

The solid obtained in step-(d) is recovered by techniques as described above.

Exemplary second solvents employed in step-(e) include, but are not limited to, an alcohol, a ketone, tetrahydrofuran, N,N-dimethylformamide, N,N-dimethylacetamide, dimethylsulfoxide, and mixtures thereof. The term solvent also includes mixtures of solvents.

Specifically, the second solvent is selected from the group consisting of methanol, ethanol, isopropyl alcohol, propanol, t-butanol, n-butanol, acetone, methyl ethyl ketone, methyl isobutyl ketone, diethyl ketone, tetrahydrofuran, N,N-dimethylformamide, N,N-dimethylacetamide, dimethylsulfoxide, and mixtures thereof; more specifically, the solvent is N,N-dimethylformamide.

In one embodiment, the dissolution in step-(e) is carried out at a temperature of about 0° C. to the reflux temperature of the solvent used, specifically at about 25° C. to about 100° C., and more specifically at about 40° C. to about 80° C.

Combining of the first solution with L-(+)-tartaric acid in step-(f) is done in a suitable order as described above, for example, the first solution is added to the L-(+)-tartaric acid, or alternatively, L-(+)-tartaric acid is added to the first solution. The addition is specifically carried out at a temperature of below about 100° C. and more specifically at a temperature of about 30° C. to about 70° C. After completion of addition process, the resulting mass is cooled and stirred at a temperature of below about 40° C. for at least 15 minutes and more specifically at about 20° C. to about 30° C. for about 20 minutes to about 5 hours.

In one embodiment, the L-(+)-tartaric acid in step-(f) is used directly or in the form of an aqueous solution of L-(+)-tartaric acid or in the form of L-(+)-tartaric acid dissolved in the second solvent as described above.

The isolation of paliperidone L-(+)-tartrate salt in step-(g) is carried out by cooling, seeding, partial removal of the solvent from the solution, by combining an anti-solvent with the solution, by substantial removal the solvent from the solution, concentrating the solution or distillation of the solvent under inert atmosphere, or a combination thereof.

Exemplary third solvents used in step-(h) include, but are not limited to, water, an alcohol, a ketone, a chlorinated hydrocarbon, a hydrocarbon, acetonitrile, tetrahydrofuran, dimethylformamide, dimethylsulfoxide, and mixtures thereof. The term solvent also includes mixtures of solvents.

Specifically, the third solvent is selected from the group consisting of water, methanol, ethanol, isopropyl alcohol, propanol, t-butanol, n-butanol, acetone, methyl ethyl ketone, methyl isobutyl ketone, diethyl ketone, methylene chloride, 1,2-dichloroethane, chloroform, carbon tetrachloride, n-pentane, n-hexane, n-heptane, cyclohexane, toluene, xylene acetonitrile, tetrahydrofuran, dimethylformamide, dimethylsulfoxide, and mixtures thereof; more specifically, the solvent is selected from the group consisting of water, methanol, ethanol, isopropyl alcohol, and mixtures thereof; and most specifically, a mixture of water and methanol.

Combining of the third solution or suspension with the reducing agent in step-(i) is done in a suitable order as described above. The addition is specifically carried out at a temperature of about 0° C. to about 90° C. for at least 10 minutes and more specifically at a temperature of about 20° C. to about 70° C. for about 30 minutes to about 4 hours. After completion of addition process, the resulting mass is stirred at a temperature of below about 100° C. for at least 5 minutes and more specifically at about 20° C. to about 70° C. for about 10 minutes to about 10 hours.

In one embodiment, the reducing agent is used directly or in the form of an aqueous solution or in the form of reducing agent dissolved in an organic solvent selected from the group as described above.

In one embodiment, the pH of the solution or suspension in step-(j) is adjusted between about 9.5 and 10.

The base used in step-(j) is selected from the group of inorganic and organic bases as described above. Specifically, the base is an inorganic base and more specifically ammonia.

In one embodiment, the ammonia used is in the form of aqueous ammonia or in the form of ammonia dissolved in an organic solvent selected from the group consisting of an alcohol and a ketone. In one embodiment, the organic solvent is selected from the group consisting of ethanol, methanol, isopropyl alcohol, and acetone.

The isolation of paliperidone free base in step-(k) is carried out, for example, by cooling, seeding, partial removal of the solvent from the solution, by combining an anti-solvent with the solution, by substantial removal the solvent from the solution, concentrating the solution or distillation of the solvent under inert atmosphere, or a combination thereof.

In one embodiment, the isolation in step-(k) is carried out by cooling the fifth solution or suspension while stiffing at a temperature of below 30° C. for at least 15 minutes, specifically at about 0° C. to about 30° C. for about 30 minutes to about 20 hours, and more specifically at about 20° C. to about 30° C. for about 1 hour to about 10 hours.

The recovery of paliperidone free base in step-(k) is accomplished by techniques such as filtration, filtration under vacuum, decantation, centrifugation, or a combination thereof. In one embodiment, the paliperidone base is recovered by filtration employing a filtration media of, for example, a silica gel or celite.

The processes for the preparation of paliperidone intermediate, 3-(2-chloroethyl)-6,7,8,9-tetrahydro-9-hydroxy-2-methyl-4H-pyrido[1,2-a]-pyrimidin-4-one, described in the prior art suffer from the drawbacks since they produce the product with lower yields and purities. The deschloro compound, 3-ethyl-6,7,8,9-tetrahydro-9-hydroxy-2-methyl-4H-pyrido[1,2-a]-pyrimidin-4-one, is formed as an impurity, in an amount of about 6% to about 10% as measured by HPLC, in the preparation of paliperidone intermediate, 3-(2-chloroethyl)-6,7,8,9-tetrahydro-9-hydroxy-2-methyl-4H-pyrido[1,2-a]-pyrimidin-4-one, when the hydrogenation reaction of 3-(2-chloroethyl)-9-hydroxy-2-methyl-4H-pyrido[1,2-a]pyrimidin-4-one is carried out using the normal grade Pd/C as a hydrogenation catalyst, and thus resulting in low overall yields of the product.

Extensive research and experimentation was carried out by the present inventors to control the formation of the deschloro impurity in the preparation of 3-(2-chloroethyl)-6,7,8,9-tetrahydro-9-hydroxy-2-methyl-4H-pyrido[1,2-a]-pyrimidin-4-one during the hydrogenation reaction. As a result, it has been found that the formation of deschloro impurity in the synthesis of 3-(2-chloroethyl)-6,7,8,9-tetrahydro-9-hydroxy-2-methyl-4H-pyrido[1,2-a]-pyrimidin-4-one can be controlled by carrying out the hydrogenation reaction of 3-(2-chloroethyl)-9-hydroxy-2-methyl-4H-pyrido[1,2-a]pyrimidin-4-one using a specific grade of Pd/C, i.e. Pd/C/854-RD or Pd/C/213-RD, as hydrogenation catalysts under suitable conditions, for example, as per the process disclosed herein.

According to another aspect, there is provided a process for the preparation of paliperidone intermediate, 3-(2-chloroethyl)-6,7,8,9-tetrahydro-9-hydroxy-2-methyl-4H-pyrido[1,2-a]-pyrimidin-4-one, with higher yield and purity, comprising:
a) providing a solution of 3-(2-chloroethyl)-9-hydroxy-2-methyl-4H-pyrido[1,2-a]pyrimidin-4-one or an acid addition salt thereof in a first solvent;
b) optionally, subjecting the solution to carbon treatment or silica gel treatment;
c) hydrogenating the solution obtained in step-(a) or step-(b) in the presence of hydrogen gas using a hydrogenation catalyst to produce a reaction mass containing 3-(2-chloroethyl)-6,7,8,9-tetrahydro-9-hydroxy-2-methyl-4H-pyrido[1,2-a]-pyrimidin-4-one, wherein the hydrogenation catalyst is Pd/C/854-RD or Pd/C/213-RD;
d) isolating 3-(2-chloroethyl)-6,7,8,9-tetrahydro-9-hydroxy-2-methyl-4H-pyrido[1,2-a]-pyrimidin-4-one as a solid from the reaction mass obtained in step-(c) using a second solvent;
e) combining the solid obtained in step-(d) with ethylenediaminetetraacetic acid (EDTA) or a salt thereof to produce a reaction mass; and
f) isolating and/or recovering substantially pure 3-(2-chloroethyl)-6,7,8,9-tetrahydro-9-hydroxy-2-methyl-4H-pyrido[1,2-a]-pyrimidin-4-one from the reaction mass obtained in step-(e).

The term “substantially pure 3-(2-chloroethyl)-6,7,8,9-tetrahydro-9-hydroxy-2-methyl-4H-pyrido[1,2-a]-pyrimidin-4-one” refers to the 3-(2-chloroethyl)-6,7,8,9-tetrahydro-9-hydroxy-2-methyl-4H-pyrido[1,2-a]-pyrimidin-4-one having purity greater than about 98%, specifically greater than about 99% and more specifically greater than about 99.5% as measured by HPLC.

Exemplary first solvents used in step-(a) include, but are not limited to, water, an alcohol, and mixtures thereof.

Specifically, the first solvent is selected from the group consisting of water, methanol, ethanol, n-propanol, isopropyl alcohol, isobutanol, n-butanol, tert-butanol, amyl alcohol, isoamyl alcohol, hexanol, and mixtures thereof; and more specifically water, methanol, and mixtures thereof.

The acid addition salt of 3-(2-chloroethyl)-9-hydroxy-2-methyl-4H-pyrido[1,2-a]pyrimidin-4-one is derived from a therapeutically acceptable acid such as hydrochloric acid, hydrobromic acid, sulfuric acid, nitric acid, oxalic acid, acetic acid, propionic acid and, phosphoric acid, succinic acid, maleic acid, fumaric acid, citric acid, glutaric acid, citraconic acid, glutaconic acid, tartaric acid, di-p-toluoyl-L-(+)-tartaric acid, malic acid, and ascorbic acid. A specific acid addition salt of 3-(2-chloroethyl)-9-hydroxy-2-methyl-4H-pyrido[1,2-a]pyrimidin-4-one is hydrochloride salt.

Step-(a) of providing a solution of 3-(2-chloroethyl)-9-hydroxy-2-methyl-4H-pyrido[1,2-a]pyrimidin-4-one or an acid addition salt thereof includes dissolving 3-(2-chloroethyl)-9-hydroxy-2-methyl-4H-pyrido[1,2-a]pyrimidin-4-one or an acid addition salt thereof in the first solvent, or obtaining an existing solution from a previous processing step.

In one embodiment, the 3-(2-chloroethyl)-9-hydroxy-2-methyl-4H-pyrido[1,2-a]pyrimidin-4-one or an acid addition salt is dissolved in the first solvent at a temperature of about 0° C. to the reflux temperature of the solvent used, specifically at about 25° C. to about 90° C., and more specifically at about 40° C. to about 80° C.

The carbon treatment or silica gel treatment in step-(b) is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate by removing charcoal or silica gel. Specifically, the finely powdered carbon is an active carbon. A specific mesh size of silica gel is 40-500 mesh, and more specifically 60-120 mesh.

In one embodiment, the hydrogenation reaction in step-(c) is carried out at a temperature of below about 0° C. to the reflux temperature of the solvent used for at least 30 minutes, specifically at a temperature of about 25° C. to about 70° C. for about 1 hour to about 4 hours, and more specifically at about 50° C. to about 60° C. for about 1 hour 30 minutes to about 2 hours 30 minutes.

Specifically, the hydrogenation catalyst used in step-(c) is 10% Pd/C/213-RD or 10% Pd/C/854-RD.

The reaction mass obtained in step-(c) is subjected to usual work-up such as washings, extractions, layer separations, evaporations, acid/base treatments, filtrations, or a combination thereof.

Exemplary second solvents used in step-(d) include, but are not limited to, water, an alcohol, a ketone, and mixtures thereof. The term solvent also includes mixtures of solvents.

Specifically, the second solvent is selected from the group consisting of water, methanol, ethanol, isopropyl alcohol, propanol, t-butanol, n-butanol, acetone, methyl ethyl ketone, methyl isobutyl ketone, diethyl ketone, and mixtures thereof; more specifically, the solvent is selected from the group consisting of water, methanol, ethanol, isopropyl alcohol, and mixtures thereof; and most specifically, water, methanol, and mixtures thereof.

In one embodiment, the isolation of 3-(2-chloroethyl)-6,7,8,9-tetrahydro-9-hydroxy-2-methyl-4H-pyrido[1,2-a]-pyrimidin-4-one in step-(d) is carried out by cooling the reaction mass while stiffing at a temperature of below 30° C. for at least 30 minutes, specifically at about 0° C. to about 30° C. for about 1 hour to about 5 hours, and more specifically at about 5° C. to about 15° C. for about 2 hours to about 4 hours.

The solid obtained in step-(d) is recovered by techniques as described above.

Exemplary salts of ethylenediaminetetraacetic acid include, but are not limited to, mono sodium, disodium, trisodium and tetrasodium salts. The ethylenediaminetetraacetic acid or its salts may be employed in a hydrated form or in an anhydrous form. A specific salt of ethylenediaminetetraacetic acid employed is ethylenediaminetetraacetic acid disodium salt dihydrate.

In one embodiment, the ethylenediaminetetraacetic acid or a salt thereof is used directly or in the form of an aqueous solution or in the form of ethylenediaminetetraacetic acid or a salt thereof dissolved in an organic solvent selected from the group consisting of an alcohol, a ketone, and mixtures thereof. Specifically, the ethylenediaminetetraacetic acid or a salt thereof is used in the form of an aqueous solution.

Combining of the solid with the ethylenediaminetetraacetic acid or a salt thereof in step-(e) is done in a suitable order as described above, for example, the solid is added to the ethylenediaminetetraacetic acid or a salt thereof, or alternatively, the ethylenediaminetetraacetic acid or a salt thereof is added to the solid. The addition is specifically carried out at a temperature of about 0° C. to about 90° C. for at least 10 minutes and more specifically at a temperature of about 20° C. to about 70° C. for about 20 minutes to about 2 hours. After completion of addition process, the resulting mass is stirred at a temperature of below about 100° C. for at least 5 minutes and more specifically at about 20° C. to about 70° C. for about 10 minutes to about 10 hours.

The reaction mass obtained in step-(e) is subjected to usual work-up such as washings, extractions, layer separations, evaporations, acid/base treatments, filtrations, or a combination thereof.

The isolation of pure 3-(2-chloroethyl)-6,7,8,9-tetrahydro-9-hydroxy-2-methyl-4H-pyrido[1,2-a]-pyrimidin-4-one in step-(f) is carried out, for example, by cooling, seeding, partial removal of the solvent from the solution, by combining an anti-solvent with the solution, by substantial removal the solvent from the solution, concentrating the solution or distillation of the solvent under inert atmosphere, or a combination thereof.

In one embodiment, the isolation in step-(f) is carried out by cooling the reaction mass while stirring at a temperature of below 30° C. for at least 15 minutes, specifically at about 0° C. to about 30° C. for about 30 minutes to about 10 hours, and more specifically at about 20° C. to about 30° C. for about 1 hour to about 5 hours.

The recovery of 3-(2-chloroethyl)-6,7,8,9-tetrahydro-9-hydroxy-2-methyl-4H-pyrido[1,2-a]-pyrimidin-4-one in step-(f) is accomplished by techniques such as filtration, filtration under vacuum, decantation, centrifugation, or a combination thereof. In one embodiment, the 3-(2-chloroethyl)-6,7,8,9-tetrahydro-9-hydroxy-2-methyl-4H-pyrido[1,2-a]-pyrimidin-4-one is recovered by filtration employing a filtration media of, for example, a silica gel or celite.

According to another aspect, there is provided a process for the preparation of 3-(2-chloroethyl)-9-hydroxy-2-methyl-4H-pyrido[1,2-a]pyrimidin-4-one hydrochloride salt with higher yield and purity, comprising:
a) reacting 2-amino-3-hydroxypyridine with 2-acetylbutyrolactone in the presence of a chlorinating agent to produce a reaction mass;
b) quenching the reaction mass in water to form a quenched reaction mass;
c) adjusting the pH of the quenched reaction mass to 4 to 6 with a base to produce a reaction mass containing 3-(2-chloroethyl)-9-hydroxy-2-methyl-4H-pyrido[1,2-a]pyrimidin-4-one;
d) extracting the reaction mass obtained in step-(c) with methylene chloride to produce an organic layer;
e) adjusting the pH of the methylene chloride layer to below 2.0 by adding concentrated hydrochloric acid to produce an acidic reaction mass;
f) substantially removing the methylene chloride from the reaction mass obtained in step-(e) to form a residue;
g) combining the residue with an alcoholic solvent to produce a solution or suspension containing 3-(2-chloroethyl)-9-hydroxy-2-methyl-4H-pyrido[1,2-a]pyrimidin-4-one hydrochloride salt; and
h) isolating and/or recovering substantially pure 3-(2-chloroethyl)-9-hydroxy-2-methyl-4H-pyrido[1,2-a]pyrimidin-4-one hydrochloride salt from the solution or suspension obtained in step-(g).

The term “substantially pure 3-(2-chloroethyl)-9-hydroxy-2-methyl-4H-pyrido[1,2-a]pyrimidin-4-one hydrochloride salt” refers to the 3-(2-chloroethyl)-9-hydroxy-2-methyl-4H-pyrido[1,2-a]pyrimidin-4-one hydrochloride salt having purity greater than about 98%, specifically greater than about 99% and more specifically greater than about 99.5% as measured by HPLC.

Exemplary chlorinating reagents used in step-(a) include, but are not limited to, thionyl chloride, phosphorus oxychloride, phosphorus trichloride and phosphorus pentachloride. A most specific chlorinating reagent is phosphorus oxychloride.

The reaction in step-(a) is carried out in presence or absence of a reaction inert solvent. Exemplary reaction inert solvents include, but are not limited to, a hydrocarbon, a chlorinated hydrocarbon, and mixtures thereof. Specifically, the reaction inert solvent is selected from the group consisting of n-pentane, n-hexane, n-heptane, cyclohexane, toluene, xylene, methylene chloride, and mixtures thereof; and a more specific reaction inert solvent is toluene or cyclohexane.

In one embodiment, the reaction in step-(a) is carried out in the presence of excess chlorinating reagent which serves as a solvent and reagent. The excess chlorinating reagent is removed from the reaction mass after completion of the reaction by distillation or may be decomposed by adding water.

The reaction in step-(a) is carried out at a temperature of about 0° C. to the reflux temperature of the solvent used, specifically at a temperature of about 25° C. to about 70° C., and more specifically at about 30° C. to about 60° C.

The base used in step-(c) is selected from the group of inorganic and organic bases as described above. Specifically, the base is an inorganic base and more specifically ammonia. In one embodiment, the ammonia is used in the form of aqueous ammonia or in the form of ammonia dissolved in an organic solvent selected from the group consisting of an alcohol and a ketone.

In one embodiment, the pH of the reaction mass in step-(c) is adjusted to about 5.3 to 5.7 while maintaining the reaction temperature at about 10° C. to about 30° C.

The reaction mass obtained step-(c) is optionally stirred at a temperature below about 50° C. for at least 15 minutes, and specifically at about 15° C. to about 30° C. for at least 30 minutes to about 3 hours.

In one embodiment, the pH of the reaction mass in step-(e) is adjusted to about 0.5 to 2.

The term “substantially removing” the solvent refers to at least 80%, specifically grater than about 85%, more specifically grater than about 90%, still more specifically grater than about 99%, and most specifically essentially complete (100%), removal of the solvent from the solvent solution.

Removal of solvent in step-(f) is accomplished, for example, by substantially complete evaporation of the solvent, concentrating the solution or distillation of solvent under inert atmosphere, or a combination thereof, to substantial elimination of total solvent present in the reaction mass.

The distillation process can be performed at atmospheric pressure or reduced pressure. Specifically, the distillation is carried out at a temperature of about 30° C. to about 110° C., more specifically at about 40° C. to about 90° C., and most specifically at about 45° C. to about 80° C.

Specifically, the solvent is removed at a pressure of about 760 mm Hg or less, more specifically at about 400 mm Hg or less, still more specifically at about 80 mm Hg or less, and most specifically from about 30 to about 80 mm Hg.

Exemplary alcohol solvents used in step-(g) is include, but are not limited to, methanol, ethanol, isopropyl alcohol, propanol, t-butanol, n-butanol, and mixtures thereof. A specific alcoholic solvent is methanol.

Combining of the residue with the alcohol solvent in step-(g) is done in a suitable order as described above, for example, the residue is added to the alcohol solvent, or alternatively, the alcohol solvent is added to the residue. The addition is specifically carried out at a temperature of about 0° C. to about 90° C. for at least 5 minutes and more specifically at a temperature of about 20° C. to about 60° C. for about 10 minutes to about 2 hours. After completion of addition process, the resulting mass is stirred at a temperature of below about 100° C. for at least 5 minutes and more specifically at about 50° C. to about 70° C. for about 10 minutes to about 5 hours.

The isolation of pure 3-(2-chloroethyl)-9-hydroxy-2-methyl-4H-pyrido[1,2-a]pyrimidin-4-one hydrochloride salt in step-(h) is carried out, for example, by cooling, seeding, partial removal of the solvent from the solution, by combining an anti-solvent with the solution, by substantial removal the solvent from the solution, concentrating the solution or distillation of the solvent under inert atmosphere, or a combination thereof.

In one embodiment, the isolation in step-(h) is carried out by cooling the reaction mass while stiffing at a temperature of below 30° C. for at least 15 minutes, specifically at about 0° C. to about 30° C. for about 30 minutes to about 10 hours, and more specifically at about 0° C. to about 10° C. for about 1 hour to about 5 hours.

The recovery of 3-(2-chloroethyl)-9-hydroxy-2-methyl-4H-pyrido[1,2-a]pyrimidin-4-one hydrochloride salt in step-(h) is accomplished by techniques such as filtration, filtration under vacuum, decantation, centrifugation, or a combination thereof. In one embodiment, the 3-(2-chloroethyl)-9-hydroxy-2-methyl-4H-pyrido[1,2-a]pyrimidin-4-one hydrochloride salt is recovered by filtration employing a filtration media of, for example, a silica gel or celite.

According to another aspect, there is provided a process for the preparation of paliperidone 9-keto impurity, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione, comprising:
a) oxidizing paliperidone with oxalyl chloride in the presence of a base in a solvent to produce a reaction mass containing 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione; and
b) isolating 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione from the reaction mass obtained in step-(a).

Exemplary solvents used in step-(a) include, but are not limited to, a ketone, a chlorinated hydrocarbon, a hydrocarbon, acetonitrile, tetrahydrofuran, N,N-dimethylformamide, N,N-dimethylacetamide, dimethylsulfoxide, and mixtures thereof. The term solvent also includes mixtures of solvents.

Specifically, the solvent is selected from the group consisting of acetone, methyl ethyl ketone, methyl isobutyl ketone, diethyl ketone, methylene chloride, 1,2-dichloroethane, chloroform, carbon tetrachloride, n-pentane, n-hexane, n-heptane, cyclohexane, toluene, xylene acetonitrile, tetrahydrofuran, N,N-dimethylformamide, N,N-dimethylacetamide, dimethylsulfoxide, and mixtures thereof; more specifically, the solvent is selected from the group consisting of methylene chloride, tetrahydrofuran, N,N-dimethylformamide, N,N-dimethylacetamide, dimethylsulfoxide, and mixtures thereof; and most specifically, methylene chloride, dimethylsulfoxide, and mixtures thereof.

The base used in step-(a) can be selected from the group of inorganic and organic bases as described above. Specifically, the base is an organic base and more specifically triethyl amine.

In one embodiment, the reaction in step-(a) is carried out at a temperature of below about 30° C., specifically at a temperature of about −80° C. to about 20° C. for at least 15 minutes, and more specifically at about −70° C. to about 0° C. for about 30 minutes to about 6 hours.

The reaction mass obtained in step-(a) is subjected to usual work-up such as washings, extractions, layer separations, evaporations, acid/base treatments, filtrations, or a combination thereof.

The isolation of 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione in step-(b) is carried out by cooling, seeding, partial removal of the solvent from the solution, by combining an anti-solvent with the solution, by substantial removal the solvent from the solution, concentrating the solution or distillation of the solvent under inert atmosphere, or a combination thereof.

The solid paliperidone 9-keto impurity obtained in step-(b) is recovered by techniques such as filtration, filtration under vacuum, decantation, centrifugation, or a combination thereof. In one embodiment, the paliperidone 9-keto impurity is recovered by filtration employing a filtration media of, for example, a silica gel or celite.

According to another aspect, there is provided a process for the preparation of paliperidone methanoyl compound (also referred to as the “methanoyl impurity”), 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one, comprising:
a) hydrogenating paliperidone using hydrogen gas in the presence of a hydrogenation catalyst and a base in a first solvent to produce a reaction mass containing 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one; and
b) isolating 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one from the reaction mass obtained in step-(a) using a second solvent.

Exemplary first solvents used in step-(a) include, but are not limited to, water, an alcohol, and mixtures thereof. The term solvent also includes mixtures of solvents.

Specifically, the solvent is selected from the group consisting of water, methanol, ethanol, isopropyl alcohol, propanol, t-butanol, n-butanol, and mixtures thereof; more specifically, the solvent is selected from the group consisting of water, methanol, ethanol, isopropyl alcohol, and mixtures thereof; and most specifically, a mixture of water and methanol.

Exemplary hydrogenation catalysts used in step-(a) include, but are not limited to, palladium hydroxide, palladium on carbon, platinum on carbon, platinum oxide, rhodium on carbon, rhodium on alumina. A specific hydrogenation catalyst is palladium on carbon.

In one embodiment, the hydrogenation reaction is carried out at a temperature of below about 50° C. for at least 30 minutes, specifically at a temperature of about −25° C. to about 40° C. for about 1 hour to about 7 hours, and more specifically at about 20° C. to about 35° C. for about 2 hours to about 5 hours.

The base used in step-(a) can be selected from the group consisting of inorganic and organic bases as described above. Specifically, the base is an inorganic base and more specifically sodium hydroxide.

The reaction mass obtained in step-(a) is subjected to usual work-up such as washings, extractions, layer separations, evaporations, acid/base treatments, filtrations, or a combination thereof.

The isolation of 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one in step-(b) is carried out by cooling, seeding, partial removal of the solvent from the solution, by combining an anti-solvent with the solution, by substantial removal the solvent from the solution, concentrating the solution or distillation of the solvent under inert atmosphere, or a combination thereof.

Exemplary second solvents used in step-(b) include, but are not limited to, water, an alcohol, a ketone, a chlorinated hydrocarbon, a hydrocarbon, acetonitrile, tetrahydrofuran, N,N-dimethylformamide, N,N-dimethylacetamide, dimethylsulfoxide, and mixtures thereof. The term solvent also includes mixtures of solvents.

Specifically, the second solvent is selected from the group consisting of water, methanol, ethanol, isopropyl alcohol, propanol, t-butanol, n-butanol, acetone, methyl ethyl ketone, methyl isobutyl ketone, diethyl ketone, methylene chloride, 1,2-dichloroethane, chloroform, carbon tetrachloride, n-pentane, n-hexane, n-heptane, cyclohexane, toluene, xylene acetonitrile, tetrahydrofuran, N,N-dimethylformamide, N,N-dimethylacetamide, dimethylsulfoxide, and mixtures thereof; more specifically, the solvent is selected from the group consisting of methylene chloride, methanol, ethanol, isopropyl alcohol, and mixtures thereof; and most specifically, a mixture of methylene chloride and methanol.

The solid paliperidone methanoyl impurity obtained in step-(b) is recovered by techniques such as filtration, filtration under vacuum, decantation, centrifugation, or a combination thereof. In one embodiment, the paliperidone methanoyl impurity is recovered by filtration employing a filtration media of, for example, a silica gel or celite.

According to another aspect, there is provided a process for the preparation of paliperidone dehydroxy compound (also referred to as the “dehydroxy impurity”), 3-[2-[4-(6-fluoro-1,2-benzisoxazol-3-yl)-1-piperidinyl]ethyl]-2-methyl-4H-pyrido[1,2-a]pyrimidin-4-one, comprising:
a) reacting 3-(2-chloroethyl)-2-methyl-4H-pyrido[1,2-a]pyrimidin-4-one with 6-fluoro-3-(4-piperidinyl)-1,2-benzisoxazole hydrochloride in the presence of N,N-diisopropylethylamine, sodium iodide and tetrabutylammonium bromide in a first solvent to produce a reaction mass containing 3-[2-[4-(6-fluoro-1,2-benzisoxazol-3-yl)-1-piperidinyl]ethyl]-2-methyl-4H-pyrido[1,2-a]pyrimidin-4-one; and
b) isolating 3-[2-[4-(6-fluoro-1,2-benzisoxazol-3-yl)-1-piperidinyl]ethyl]-2-methyl-4H-pyrido[1,2-a]pyrimidin-4-one from the reaction mass obtained in step-(a) using a second solvent.

Exemplary first solvents used in step-(a) include, but are not limited to, an alcohol, a ketone, a chlorinated hydrocarbon, a hydrocarbon, acetonitrile, tetrahydrofuran, N,N-dimethylformamide, N,N-dimethylacetamide, dimethylsulfoxide, and mixtures thereof. The term solvent also includes mixtures of solvents.

Specifically, the first solvent is selected from the group consisting of methanol, ethanol, isopropyl alcohol, propanol, t-butanol, n-butanol, acetone, methyl ethyl ketone, methyl isobutyl ketone, diethyl ketone, methylene chloride, 1,2-dichloroethane, chloroform, carbon tetrachloride, n-pentane, n-hexane, n-heptane, cyclohexane, toluene, xylene acetonitrile, tetrahydrofuran, N,N-dimethylformamide, N,N-dimethylacetamide, dimethylsulfoxide, and mixtures thereof; more specifically, the solvent is selected from the group consisting of methanol, ethanol, isopropyl alcohol, and mixtures thereof; and a most specific solvent is methanol.

In one embodiment, the condensation reaction in step-(a) is carried out at a temperature of about 0° C. to the reflux temperature of the solvent used, specifically at a temperature of about 20° C. to about 80° C. for at least 10 hours, and more specifically at about 50° C. to about 70° C. for about 20 hours to about 75 hours.

The reaction mass obtained in step-(a) is subjected to usual work-up such as washings, extractions, layer separations, evaporations, acid/base treatments, filtrations, or a combination thereof.

The isolation of 3-[2-[4-(6-fluoro-1,2-benzisoxazol-3-yl)-1-piperidinyl]ethyl]-2-methyl-4H-pyrido[1,2-a]pyrimidin-4-one in step-(b) is carried out by cooling, seeding, partial removal of the solvent from the solution, by combining an anti-solvent with the solution, by substantial removal the solvent from the solution, concentrating the solution or distillation of the solvent under inert atmosphere, or a combination thereof.

Exemplary second solvents used in step-(b) include, but are not limited to, water, an alcohol, a ketone, a chlorinated hydrocarbon, a hydrocarbon, acetonitrile, tetrahydrofuran, N,N-dimethylformamide, N,N-dimethylacetamide, dimethylsulfoxide, and mixtures thereof. The term solvent also includes mixtures of solvents.

Specifically, the second solvent is selected from the group consisting of water, methanol, ethanol, isopropyl alcohol, propanol, t-butanol, n-butanol, acetone, methyl ethyl ketone, methyl isobutyl ketone, diethyl ketone, methylene chloride, 1,2-dichloroethane, chloroform, carbon tetrachloride, n-pentane, n-hexane, n-heptane, cyclohexane, toluene, xylene acetonitrile, tetrahydrofuran, N,N-dimethylformamide, N,N-dimethylacetamide, dimethylsulfoxide, and mixtures thereof; more specifically, the solvent is selected from the group consisting of water, methanol, ethanol, isopropyl alcohol, and mixtures thereof; and most specifically, a mixture of water and methanol.

The solid paliperidone dehydroxy impurity obtained in step-(b) is recovered by techniques such as filtration, filtration under vacuum, decantation, centrifugation, or a combination thereof. In one embodiment, the paliperidone dehydroxy impurity is recovered by filtration employing a filtration media of, for example, a silica gel or celite.

Further encompassed herein is the use of the highly pure paliperidone or a pharmaceutically acceptable salt thereof substantially free of methanoyl, dehydroxy and 9-keto impurities for the manufacture of a pharmaceutical composition together with a pharmaceutically acceptable carrier.

specific pharmaceutical composition of highly pure paliperidone or a pharmaceutically acceptable salt thereof substantially free of methanoyl, dehydroxy and 9-keto impurities is selected from a solid dosage form and an oral suspension.

In one embodiment, the highly pure paliperidone or a pharmaceutically acceptable salt thereof substantially free of methanoyl, dehydroxy and 9-keto impurities has a D₉₀ particle size of less than or equal to about 400 microns, specifically about 1 micron to about 300 microns, and most specifically about 10 microns to about 150 microns.

In another embodiment, the particle sizes of the highly pure paliperidone or a pharmaceutically acceptable salt thereof substantially free of methanoyl, dehydroxy and 9-keto impurities are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from psychotic diseases such as schizophrenia, comprising administering a therapeutically effective amount of the highly pure paliperidone or a pharmaceutically acceptable salt thereof substantially free of methanoyl, dehydroxy and 9-keto impurities, or a pharmaceutical composition that comprises a therapeutically effective amount of highly pure paliperidone or a pharmaceutically acceptable salt thereof substantially free of methanoyl, dehydroxy and 9-keto impurities, along with pharmaceutically acceptable excipients.

According to another aspect, there is provided pharmaceutical compositions comprising highly pure paliperidone or a pharmaceutically acceptable salt thereof substantially free of methanoyl, dehydroxy and 9-keto impurities prepared according to the processes disclosed herein and one or more pharmaceutically acceptable excipients.

According to another aspect, there is provided a process for preparing a pharmaceutical formulation comprising combining highly pure paliperidone or a pharmaceutically acceptable salt thereof substantially free of methanoyl, dehydroxy and 9-keto impurities prepared according to processes disclosed herein, with one or more pharmaceutically acceptable excipients.

Yet in another embodiment, pharmaceutical compositions comprise at least a therapeutically effective amount of highly pure paliperidone or a pharmaceutically acceptable salt thereof substantially free of methanoyl, dehydroxy and 9-keto impurities. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure paliperidone or a pharmaceutically acceptable salt thereof substantially free of methanoyl, dehydroxy and 9-keto impurities may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure paliperidone or a pharmaceutically acceptable salt thereof substantially free of methanoyl, dehydroxy and 9-keto impurities within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### EXPERIMENTAL

### High Performance Liquid Chromatography (HPLC)

The HPLC purity was measured by high performance liquid chromatography by Water's HPLC system having alliance 2695 model pump and 2487 (UV) detector with Empower chromatography software or its equivalent maintaining following conditions:

### Column: UNISON UK C18 (250×4.6) mm, 5.0μ Make: Imkat

### Detector: UV at 237 nm

Flow rate: 1.0 mL/min
Injection volume: 10.0 μL
Run time: 60 min
Oven temperature: 40° C.
Diluent: water:Acetonitrile (50:50% v/v)

### Elution: Gradient

Buffer: Ammonium dihydrogen phosphate with pH 4.6 (±0.05).
Mobile Phase-A: Buffer and Methanol (85:15% v/v)
Mobile Phase-B: Buffer and Methanol (35:65% v/v)

### Gradient Program:

| Time (min) | (%) Mobile phase-A | (%) Mobile phase-B |
|---|---|---|
| 0 | 100 | 0 |
| 35 | 0 | 100 |
| 55 | 0 | 100 |
| 56 | 100 | 0 |
| 62 | 100 | 0 |

### COMPARATIVE EXAMPLES

### Comparative Example 1

### Preparation of 3-(2-Chloroethyl)-9-hydroxy-2-methyl-6,7,8,9-tetrahydro-4H-pyrido[1,2-a]pyrimidin-4-one using normal 10% palladium on carbon

A mixture of 3-(2-chloroethyl)-9-hydroxy-2-methyl-4H-pyrido[1,2-a]pyrimidin-4-one hydrochloride (250 g) and methanol (2175 ml) was heated at 50-55° C., followed by addition of activated carbon (62.5 g) and stiffing for 1 hour. The resulting clear solution was filtered through a hyflo bed and the bed was washed with methanol (250 ml). The filtrate was taken an autoclave equipped with gas a spurger, stirrer, and thermo pocket and then inertized with nitrogen gas, followed by the addition of palladium on carbon (10% Pd, 55 g). The autoclave was flushed with nitrogen and the resulting suspension was heated at 40-45° C., followed by slow maintenance of 0.5 kg/cm² pressure of hydrogen gas under stiffing for 14 hours. The reaction mass, obtained after completion of reaction, was unloaded from autoclave. The reaction mixture was filtered under nitrogen gas, and the hyflo bed was washed with methanol (250 ml). The methanol was evaporated from the filtrate at 50-55° C. under reduced pressure to obtain a thick oil, followed by the addition of water (2500 ml) at 25-30° C. Production of the clear solution was followed by the drop wise addition of a solution of sodium acetate (187 g) dissolved in water (500 ml) over 1 hour followed by stirring for 15 minutes. Further, the slurry was stirred for 2 hours at 0-5° C. The crystals were filtered, washed with water (2×500 ml) to provide the wet solid. The wet solid was dried under reduced pressure to produce 105 g of 3-(2-chloroethyl)-9-hydroxy-2-methyl-6,7,8,9-tetrahydro-4H-pyrido[1,2-a]pyrimidin-4-one (Yield: 47.7%; HPLC purity: 90.56%; and Content of deschloro impurity: 6.41%).

### Comparative Example 2

### Purification of Paliperidone without Using Sodium Dithionite

Crude paliperidone (10 g) was suspended in a mixture of methanol (50 ml) and water (50 ml) under stirring. The pH of the resulting mass was adjusted to 3 to 3.5 by adding a solution of 10% hydrochloric acid at 25-30° C., followed by stirring for 5 minutes. The resulting clear solution was basified by adjusting the pH to 5.5 to 6.0 with aqueous methanolic ammonia solution at 25-30° C. The resulting mass was heated at 50-55° C., followed by the addition of activated carbon (0.5 g) and stiffing for 10-15 minutes. The resulting solution was filtered through a hyflo bed and the bed was washed with a mixture of water (10 ml) and methanol (10 ml). The filtrate and washings were combined, followed by adjusting the pH 8.5 with 5% aqueous ammonia solution at 25-30° C. The resulting slurry was stirred at 25-30° C. for 30 minutes. The crystals were isolated by filtration and washing with purified water (2×50 ml) and acetone (2×10 ml). The wet material was suspended in acetone (100 ml), followed by stirring at 50-55° C. The slurry was cooled to 25-30° C. followed by stirring for 30 minutes. The crystals were isolated by filtration and washed with acetone (10 ml). The wet material was dried in the oven at 45-50° C. under reduced pressure to yield 8.2 g of pure paliperidone (HPLC purity: 99.61%, Content of 9-Keto impurity: 0.17%).

### Comparative Example 3

### Preparation of Pure Paliperidone According to the Process Described in Example 1 of Pct Publication No. WO 2009/118655

### Step-I: Preparation of Crude Paliperidone

The mixture of 3-(2-chloroethyl)-6,7,8,9-tetrahydro-9-hydroxy-2-methyl-4H-pyrido[1,2-a]-pyrimidin-4-one (60 g), 6-fluoro-3-(4-piperidinyl)-1,2-benzisoxazole (48.4 g), sodium carbonate (91.87 g) and ethanol (1200 ml) was stirred for 24 hours at 60-65° C. under a nitrogen atmosphere. After completion of the reaction, the reaction mass was filtered and then ethanol was distilled off completely. The product was extracted with methylene chloride (1000 ml) and the methylene chloride layer was washed with water and then dried over sodium sulphate. Methylene chloride was distilled out completely, the product was diluted with methanol (200 ml), and then stirred for 2-3 hours at 30-35° C. The solid obtained was isolated by filtration, washed with methanol (50 ml) and then dried under vacuum at 40-45° C. to produce 50 g of crude paliperidone (Content of the keto impurity: 0.53 wt % as measured by HPLC).

### Step-II: Purification of Paliperidone

Crude paliperidone (50 g, obtained in step-I) was stirred with dimethylformamide (250 ml) at 50-60° C. for 3 hours and the resulting mass was cooled to 25° C. The product was then filtered followed by washings with dimethylformamide (50 ml) and methanol (50 ml). The wet solid was stirred with dimethylformamide (150 ml) for 3 hours at 50-60° C., the resulting mass was cooled to 25° C. and then filtered. The filtered solid was washed with dimethylformamide (50 ml) followed by methanol (50 ml). The resulting wet product was stirred with methanol (250 ml) for 3 hours at 30° C., and the solid was filtered and washed with methanol to produce paliperidone (Wet weight: 28 g). The product was then added to methanol (1800 ml) and the resulting mixture was heated to 65° C. under stirring to get a clear solution. The solution was cooled to 50° C. followed by addition of activated carbon (7.5 g) and then the mixture was stirred for 30 minutes at 50° C. The resulting hot solution was filtered through hyflo to remove charcoal. Sodium borohydride (25 mg) was added into filtrate and stirred for 2 hours at 50° C. The solution was then concentrated to one-fourth volume under vacuum and then cooled to 25° C. The separated solid was filtered, washed with methanol and then dried to produce 24 g of pure paliperidone (HPLC Purity: 99.85%; Content of the keto impurity: 0.05 wt %).

The following examples are given for the purpose of illustrating the present disclosure and should not be considered as limitation on the scope or spirit of the disclosure.

### EXAMPLES

### Example 1

### Preparation of 3-(2-Chloroethyl)-9-hydroxy-2-methyl-4H-pyrido[1,2-a]pyrimidin-4-one hydrochloride

2-Amino-3-hydroxypyridine (350 g, 3.18 mol) was added to phosphorus oxychloride (1463 g, 9.54 mol) in a clean and dry reaction assembly under stiffing over a period of 1 hour at 30-35° C. and followed by addition of 2-acetylbutyrolactone (490 g, 3.82 mol). The resulting mass was stirred for about 24 hours at 35-40° C., followed by quenching the mass in water (3500 ml) and maintaining the temperature at 15-30° C. After completion of quenching, the reaction assembly was flushed with water (350 ml) and transferred to the quenched mass. The pH of the reaction mass was adjusted to 5.3 to 5.7 using 20-25% aqueous ammonia solution while maintaining the temperature at 15-25° C. and the resulting mass was stirred for 30 minutes at 20-25° C. The reaction mass was extracted three times with dichloromethane (2800 ml+1750 ml+1750 ml) and the combined dichloromethane extract was washed with water (1400 ml), followed by filtration of dichloromethane layer through a hyflo bed. The pH of the dichloromethane filtrate was adjusted to below 2.0 by adding concentrated hydrochloric acid followed by distillation of acidic dichloromethane at atmospheric pressure until the temperature reached 50-55° C. Further distillation was continued under reduced pressure at 50-55° C. The resulting mass was co-distilled two times with methanol (2×350 ml) under reduced pressure. Methanol (700 ml) was added to the resulting mass and the temperature was raised to 60-65° C. followed by stirring for 15 minutes. The mass was then gradually cooled to 20-25° C. followed by further stiffing for 5 hours. The resulting mass was further cooled to 0-5° C. and maintained for 2 hours. The product was isolated by filtration followed by washing with chilled methanol (350 ml). The resulting wet cake was dried under reduced pressure at 50-55° C. to produce 640 g of 3-(2-chloroethyl)-9-hydroxy-2-methyl-4H-pyrido[1,2-a]pyrimidin-4-one hydrochloride (Yield: 73.2%; HPLC Purity: 99.5%).

¹H-NMR (DMSO, δ): 2.64 (3H, s), 3.09 (2H, t), 3.80 (2H, t), 7.54 (1H, t), 7.81 (1H, d), 8.61 (1H, d).

### Example 2

### Preparation of 3-(2-Chloroethyl)-9-hydroxy-2-methyl-6,7,8,9-tetrahydro-4H-pyrido[1,2-a]pyrimidin-4-one

3-(2-Chloroethyl)-9-hydroxy-2-methyl-4H-pyrido[1,2-a]pyrimidin-4-one hydrochloride (400 g, 1.4538 mol) and methanol (4800 ml) were placed in a 10 L reaction flask equipped with gas spurger, stirrer and thermo pocket. The resulting solution was inertized with nitrogen gas followed by addition of palladium on carbon (10% Pd/C/RD-854, 80 g). The resulting suspension was heated at 50-55° C. followed by slow bubbling of hydrogen gas under stiffing for 2 hours. After completion of the reaction, the reaction mass was cooled to 25-30° C., followed by the addition of thiophene (4 ml) and then removing the catalyst by filtration through a hyflo bed under nitrogen gas. The filtration bed was washed with methanol (2×400 ml) and then the methanol was evaporated from the filtrate at 50-55° C. under reduced pressure to obtain a thick oil. Water (1480 ml) was added to the resulting oil followed by heating at 80-85° C. for 15 minutes. The resulting solution was cooled to 25-30° C. and then treated with a solution of potassium acetate (285.4 g, 2.9077 mol) in water (300 ml) drop wise over 1 hour. The resulting mass was stirred for 1 hour at 25-30° C., followed by cooling to 8-12° C. and further maintaining for 2 hours. The resulted crystals were filtered, washed with aqueous solution of ethylenediaminetetraacetic acid disodium salt dihydrate (2 g, dissolved in 400 ml of water), followed by water (400 ml) and chilled isopropyl alcohol (200 ml) to obtain the wet solid. The wet solid was dried under reduced pressure to produce 266 g of 3-(2-chloroethyl)-9-hydroxy-2-methyl-6,7,8,9-tetrahydro-4H-pyrido[1,2-a]pyrimidin-4-one (Yield: 75.4%; HPLC purity: 99.4%; and Content of Deschloro impurity: 0.4%).

### Example 3

### Preparation of 3-(2-Chloroethyl)-9-hydroxy-2-methyl-6,7,8,9-tetrahydro-4H-pyrido[1,2-a]pyrimidin-4-one

A mixture of 3-(2-chloroethyl)-9-hydroxy-2-methyl-4H-pyrido[1,2-a]pyrimidin-4-one hydrochloride (500 g, 1.82 mol), activated carbon (100 g), and methanol (5000 ml) was heated at 50-55° C., followed by stirring for 1 hour. The resulting clear solution was filtered through hyflo bed and the bed was washed with methanol (500 ml). The filtrate was taken into a separate reaction flask equipped with gas spurger, stirrer, and thermo pocket and then inertized with nitrogen gas followed by the addition of palladium on carbon (10% Pd/C/RD-213, 100 g). The resulting suspension was heated at 50-55° C., followed by slow bubbling of hydrogen gas under stiffing for 2 hours. After completion of reaction, thiophene (3 g) was added to the resulting mass. The reaction mixture was filtered under nitrogen gas, and the hyflo bed was washed with methanol (500 ml). The methanol was evaporated from the filtrate at 50-55° C. under reduced pressure to obtain a thick oil, followed by the addition of water (1850 ml) at 80-85° C. The mixture was cooled to 25-30° C., followed by the drop wise addition of a solution of potassium acetate (356.6 g, 3.64 mol) in water (364 ml) over 1 hour and stiffing for 3 hours at 5-7° C. The crystals were filtered, washed with water (2×500 ml) to provide the wet solid. The wet solid was further recrystallized according to any one of the following two methods:

### Crystallization Method A:

The wet solid was crystallized from acetone to obtain 275 g (62.35%) of 3-(2-chloroethyl)-9-hydroxy-2-methyl-6,7,8,9-tetrahydro-4H-pyrido[1,2-a]pyrimidin-4-one (HPLC purity: 98.5%).

### Crystallization Method B:

The wet solid was crystallized using isopropyl alcohol to obtain 280 g (63.5%) of 3-(2-chloroethyl)-9-hydroxy-2-methyl-6,7,8,9-tetrahydro-4H-pyrido[1,2-a]pyrimidin-4-one (HPLC purity: 99%).

### Example 4

### Preparation of Paliperidone

3-(2-Chloroethyl)-6,7,8,9-tetrahydro-9-hydroxy-4H-pyrido[1,2-a]pyrimidin-4-one (175 g, 0.7211 moles, obtained in Example 2), 6-fluoro-3-(4-piperidinyl)-1,2-benzisoxazole hydrochloride (184.41 g, 0.7211 moles), sodium iodide (8.75 g), tetrabutylammonium bromide (8.75 g), N,N-diisopropylethylamine (372.1 g, 2.8844 moles) and methanol (1750 ml) were taken into a four-necked RB flask equipped with stirrer, condenser, gas inlet tube and thermo pocket under nitrogen atmosphere at 25-30° C. The contents were heated at 55-60° C. and maintained for 40 hours under stiffing. After completion of reaction, the mass was cooled to 25-30° C., followed by stiffing the mass for 2 hours at 25-30° C. and then filtering. The wet cake was washed with methanol (2×260 ml) and suction dried. The resulting wet cake was slurried in water (2000 ml) for 30 minutes followed by isolation of the solid by filtration and subsequent washings with water (2×350 ml) and N,N-dimethylformamide (175 ml). The resulting wet cake was dissolved in N,N-dimethylformamide (1750 ml) at 85-90° C. and stirred for 10 minutes to provide a clear solution. The resulting solution was cooled to 55-60° C. followed by the addition of L-(+)-tartaric acid (43.3 gm) solution in N,N-dimethylformamide (175 ml) under stirring. The mixture was further cooled to 25-30° C. and maintained for 30 minutes. The resulting mass was filtered, and the wet cake was washed with N,N-dimethylformamide (175 ml) and methanol (260 ml). The wet cake was suspended in a mixture of methanol (1575 ml) and water (175 ml), followed by the addition of an aqueous solution of sodium dithionite (1.75 g dissolved in 35 ml of water). The pH of suspension was adjusted to 9.5 to 10 using 25% aqueous ammonia solution at 25-30° C. The resulting suspension was stirred for 60 minutes, the separated solid was filtered, washed with water (2×350 ml) followed by methanol (2×260 ml) and then suck dried. The wet cake was dried in the oven at 50-55° C. under reduced pressure to yield 212.5 g of paliperidone (HPLC purity: 99.86%).

Content of Impurities: 9-Keto impurity: 0.07%; Methanoyl impurity: Below detection limit; and Dehydroxy impurity: 0.03%.

### Example 5

### Preparation of Crude Paliperidone

A mixture of 6-fluoro-3-(4-piperidinyl)-1,2-benzisoxazole hydrochloride (105.37 g, 0.412 moles), 3-(2-chloroethyl)-2-methyl-6,7,8,9-tetrahydro-4H-pyrido[1,2-a]pyrimidine-4-one hydrochloride (100 g, 0.412 moles, obtained in Example 3), dry powdered sodium carbonate (174.68 g, 1.648 moles) and dry methanol (1000 ml) was placed in a pressure vessel under nitrogen atmosphere and the mixture was stirred for 5-6 hours at 70-75° C. After completion of reaction, the pressure was brought to atmospheric level and the reaction mass was concentrated under reduced pressure. Acetone (500 ml) was added to the concentrated mass followed by stirring for 1 hour, the mass was filtered and the resulting wet solid was washed with acetone (100 ml). The wet cake was washed with water (1000 ml) followed by stiffing for 30 minutes. The resulting solid was isolated by filtration and washed with water (2×200 ml) and methanol (2×200 ml). The resulting wet cake was dissolved in N,N-dimethylformamide (1000 ml) at 90-95° C. and stirred for 10 minutes to provide a clear solution. The resulting solution was cooled to 20-25° C. and stirred for 3 hours. The resulting mass was filtered and the wet cake was washed with N,N-dimethylformamide (100 ml) and methanol (2×100 ml). The resulting wet material was dried in the oven at 50-55° C. under reduced pressure to yield 107 g of crude paliperidone (Yield: 61.0%; HPLC Purity: 99.02%).

Content of Impurities: 9-Keto impurity: 0.28%; Methanoyl impurity: 0.41%; and Dehydroxy impurity: Below detection limit.

### Example 6

### Purification Paliperidone

Paliperidone (175 g, obtained in Example 4) was suspended in a mixture of methanol (750 ml) and water (750 ml) under stirring. The pH of the resulting mass was adjusted to 3 to 3.5 by adding a solution of concentrated hydrochloric acid (31.25 ml) diluted with methanol (281.25 ml) at 30-35° C. The resulting clear solution was basified by adjusting the pH to 5.5 to 6.0 with methanolic ammonia solution at 30-35° C. The resulting mass was maintained for 5 minutes at 30-35° C., followed by the addition of an aqueous solution of sodium dithionite (prepared by dissolving 3.75 g of sodium dithionite in 75 ml of water) and stirring the mass for 30 minutes at 30-35° C. The production of the solution was followed by the addition of activated carbon (7.5 g, type: Norit DX ultra neutral) and stirred for 30 minutes. The resulting solution was filtered through a hyflo bed and the bed was washed with a mixture of water (75 ml) and methanol (75 ml). The filtrate and washings were combined, followed by adjusting the pH 8.5 to 9 with methanolic ammonia solution at 30-35° C. The resulting slurry was heated at 35-40° C. and maintained for 15 minutes. The resulting slurry was cooled to 25-30° C. and maintained for 1 hour and filtered. The wet cake was slurried in water (750 ml) by stiffing for 30 minutes followed by isolation of the solid by filtration and washing with purified water (2×300 ml) and methanol (2×225 ml). The wet material was dried in the oven at 45-50° C. under reduced pressure to yield 138.3 g of pure paliperidone (HPLC purity: 99.89%).

Content of Impurities: 9-keto impurity: 0.05%; Methanoyl impurity: Below detection limit; and Dehydroxy impurity: Below detection limit.

### Example 7

### Purification of Paliperidone

Crude paliperidone (50 g, obtained in Example 5) was suspended in a mixture of methanol (250 ml) and water (250 ml) under stirring. The pH of the resulting mass was adjusted to 3 to 3.5 by adding a solution of concentrated hydrochloric acid (12 ml) diluted with methanol (108 ml) at 20-25° C. The resulting clear solution was basified by adjusting the pH to 5.5 to 6.0 with aqueous diluted ammonia solution at 20-25° C. The resulting mass was heated at 50-55° C. and maintained for 30 minutes followed by the addition of 1% aqueous solution of sodium dithionite (62.63 ml) and stiffing the mass for 30 minutes at 50-55° C. The second portion of 1% aqueous solution of sodium dithionite (62.63 ml) was added to the resulting mass and then stirred for 30 minutes. The hot solution was followed by the addition of activated carbon (2.5 g) and stirred for 30 minutes. The resulting solution was filtered through hyflo bed and washed the bed with water (50 ml) and methanol (50 ml). The filtrate and washings were combined and followed by adjusting the pH 8.5 to 9 with aqueous diluted ammonia solution at 20-25° C. The resulting slurry was stirred for 1 hour and filtered. The wet cake was washed with water (250 ml) by stiffing for 30 minutes followed by isolation of the solid by filtration and washing with water (2×100 ml) and acetone (2×50 ml). The resulting wet cake was suction dried and suspended in acetone (500 ml) followed by refluxing the slurry for 30 minutes. The resulting slurry was cooled, filtered and the wet cake was washed with acetone (50 ml). The wet material was dried in the oven at 45-50° C. under reduced pressure to yield 44 g of pure paliperidone (HPLC purity 99.9%).

Content of Impurities: 9-keto impurity: 0.07%; Methanoyl impurity: 0.01%; and Dehydroxy impurity: 0.01%.

### Example 8

### Purification of Paliperidone

Paliperidone (25 g, obtained in Example 4) was suspended in a mixture of isopropyl alcohol (125 ml) and water (125 ml) under stirring. The pH of the resulting mass was adjusted to 3 to 3.5 by adding a solution of concentrated hydrochloric acid (5 ml) diluted with isopropyl alcohol (45 ml) at 30-35° C. The resulting clear solution was basified by adjusting the pH to 5.5 to 6.0 with aqueous diluted ammonia solution at 20-25° C. The resulting mass was maintained for 15 minutes at 30-35° C. followed by the addition of an aqueous solution of sodium dithionite (prepared by dissolving 0.625 g of sodium dithionite in 62.5 ml of water) and stirring the mass for 30 minutes at 30-35° C. The solution was followed by the addition of activated carbon (1.25 g, type: Norit DX ultra neutral) and stirred for 30 minutes. The resulting solution was filtered through a hyflo bed and the bed was washed with water (25 ml) and isopropyl alcohol (25 ml). The filtrate and washings were combined and followed by adjusting the pH 8.5 to 9 with aqueous diluted ammonia solution at 25-30° C. The resulting slurry was stirred for 1 hour and filtered. The wet cake was washed with water (125 ml) by stiffing for 30 minutes followed by isolation of the solid by filtration and washing with water (50 ml) and isopropyl alcohol (2×25 ml). The resulting wet cake was suction dried and suspended in isopropyl alcohol (250 ml), followed by stirring the slurry for 15-20 minutes at 50-55° C. The resulting slurry was cooled, filtered and the wet cake was washed with isopropyl alcohol (25 ml). The wet material was dried in the oven at 45-50° C. under reduced pressure to yield 21 g of pure paliperidone (HPLC purity 99.84%).

Content of Impurities: 9-keto impurity: 0.04%; Methanoyl impurity: 0.02%; and Dehydroxy impurity: 0.04%.

### Example 9

### Preparation of 3-[2-[4-[1-(4-Fluoro-2-hydroxyphenyl)methanoyl]piperidinyl-1-yl]ethyl]-2-methyl-6,7,8,9-tetrahydro-4H-pyrido[1,2-a]pyrimidin-4-one (methanoyl compound or methanoyl impurity)

Methanol (2125 ml), paliperidone (25 g, 0.0586 moles), sodium hydroxide (2.81 g, 0.07025 moles), water (1.5 ml) and 10% palladium on carbon (30 g) were taken into a four neck round bottom flask equipped with condenser, thermo pocket, gas bubbler and stirrer under nitrogen atmosphere at 25-30° C. Hydrogen gas was bubbled into the resulting suspension and continued for 2 hours at 30 to 35° C. After completion of the reaction, the mass was filtered and the bed was washed with methanol (2×100 ml). The methanol was completely evaporated from the filtrate. Methanol (500 ml) was added to the residue and acidified by adjusting the pH to 2.5 to 3.0 using concentrated hydrochloric acid. The resulting acidic mass was basified to pH 5.0 to 5.5 using ammonia solution followed stiffing for 30 minutes at 25 to 30° C. The solid was isolated by filtration followed by washing with methanol (2×25 ml). The resulting solid was dissolved in a mixture of dichloromethane: methanol (500 ml, 1:1) at 50-55° C. The resulting solution was filtered through a hyflo bed and the solvent was evaporated under reduced pressure at 40-55° C. till to get the product in slurry form. The resulting slurry was cooled to 15-20° C. and isolated the solid by filtration. The resulting solid was dried under reduced pressure at 50-55° C. to yield 3.6 g of 3-[2-[4-[1-(4-Fluoro-2-hydroxyphenyl)methanoyl]piperidinyl-1-yl]ethyl]-2-methyl-6,7,8,9-tetrahydro-4H-pyrido[1,2-a]pyrimidin-4-one.

### Example 10

### Preparation of 3-[2-[4-(6-Fluoro-1,2-benzisoxazol-3-yl)-1-piperidinyl]ethyl]-2-methyl-4H-pyrido[1,2-a]pyrimidin-4-one (Dehydroxy compound or Dehydroxy impurity)

Methanol (150 ml), 3-(2-chloroethyl)-2-methyl-4H-pyrido[1,2-a]pyrimidin-4-one (15 g, 0.0674 moles), 6-fluoro-3-(4-piperidinyl)-1,2-benzisoxazole hydrochloride (18.53 g, 0.0721 moles), N,N-diisopropylethylamine (28 g, 0.217 moles), sodium iodide (0.75 g) and tetrabutylammonium bromide (0.75 g) were taken into a four neck round bottom flask equipped with condenser, thermo pocket, nitrogen inlet and stirrer at 25-30° C. The resulting mixture was heated at 60-65° C. and maintained for 3 days. The resulting suspension was cooled to 30-35° C. and the solid was isolated by filtration. The wet cake was washed with water (100 ml) and methanol (2×30 ml). The resulting solid was dried under reduced pressure at 50-55° C. to yield 20 g of 3-[2-[4-(6-Fluoro-1,2-benzisoxazol-3-yl)-1-piperidinyl]ethyl]-2-methyl-4H-pyrido[1,2-a]pyrimidin-4-one (HPLC purity 99.74%).

H¹ NMR (8, in DMSO): 1.82-1.85 (qq, 2H), 2.02-2.05 (bd, 2H), 2.17-2.22 (t, 2H), 2.45-2.51 (m, 6H), 2.78-2.82 (t, 2H), 3.05-3.13 (m, 3H), 7.23-7.28 (m, 2H), 7.54 (d, 1H), 7.66-7.68 (dd, 1H), 7.81-7.85 (m, 1H), 7.98-8.01 (dd, 1H) and 8.86 (d, 1H); Mass [M+H]: 407.10.

### Example 11

### Preparation of 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione (9-keto impurity)

Dichloromethane (1750 ml) and oxalyl chloride (44.64 g, 0.3516 moles) were taken into a four necked round bottom flask equipped with condenser, thermo pocket, nitrogen inlet and stirrer under stirring at 25-30° C. The resulting solution was cooled to −65° C. to −70° C. followed by slow addition of dimethylsulfoxide (54.94 g, 0.703 moles) at −65° C. to −70° C. over a period of 15 minutes and stirring for 15 minutes at −65° C. to −70° C. A solution of paliperidone (50 g, 0.1172 moles) dissolved in dichloromethane (2000 ml) was added to the resulting solution at −65° C. to −70° C. over a period of 30 minutes followed by stirring for 15 minutes at −65° C. to −70° C. Triethyl amine (71.16 g, 0.703 mole) was added to the reaction mass at −65° C. to −70° C. over a period of 15 minutes. The temperature of reaction mass was raised to −5 to 0° C. and then stirred for 1 hour at −5 to 0° C. After completion of reaction, saturated ammonium chloride solution (500 ml) was added to the reaction mass and stirred for 10 minutes. The layers were allowed for separation and lower dichloromethane layer was separated. The resulting dichloromethane layer was washed with saturated ammonium chloride (2×500 ml) and water (2×500 ml) followed by drying of the dichloromethane layer over sodium sulfate. The dichloromethane was evaporated under vacuum while maintaining temperature at below 40° C. The resulting residue was triturated twice with dichloromethane (125 ml+50 ml) followed by isolation of yellow solid. The resulting solid was dried under reduced pressure at 40-45° C. to yield 14 g of 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione.

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP).

The term “crude paliperidone or a pharmaceutically acceptable salt thereof” as used herein refers to paliperidone or a pharmaceutically acceptable salt thereof containing greater than about 0.1 area-%, more specifically greater than about 0.15 area-%, still more specifically greater than about 0.2 area-% and most specifically greater than about 0.3 area-% of at least one, or more, of the methanoyl, dehydroxy and 9-keto impurities.

As used herein, the term, “detectable” refers to a measurable quantity measured using an HPLC method having a detection limit of 0.01 area-%.

As used herein, in connection with amount of impurities in paliperidone or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a processor method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent.

The important characteristics of the PSD were the (D₉₀), which is the size, in microns, below which 90% of the particles by volume are found, and the (D₅₀), which is the size, in microns, below which 50% of the particles by volume are found.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Paliperidone or a pharmaceutically acceptable salt thereof comprising a 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one impurity (methanoyl impurity) in an amount of less than 0.1 area-% as measured by HPLC.

2. Paliperidone of claim 1, comprising the methanoyl impurity in an amount of about 0.01 area-% to about 0.1 area-%; wherein the paliperidone has a total purity of about 99% to about 99.99% as measured by HPLC; wherein the paliperidone further comprises one, or both, of a 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione impurity (9-keto impurity) and a 3-[2-[4-(6-fluoro-1,2-benzisoxazol-3-yl)-1-piperidinyl]ethyl]-2-methyl-4H-pyrido[1,2-a]pyrimidin-4-one impurity (dehydroxy impurity), each, in an amount of less than 0.1 area-% as measured by HPLC; and wherein the pharmaceutically acceptable salt of paliperidone is a hydrochloride salt, a hydrobromide salt, an oxalate salt, a nitrate salt, a sulfate salt, a phosphate salt, a fumarate salt, a succinate salt, a maleate salt, a besylate salt, a tosylate salt, a palmitate salt or a tartrate salt.

3.-7. (canceled)

8. A purification process for obtaining highly pure paliperidone or a pharmaceutically acceptable salt thereof of claim 1, comprising:
a) providing a first solution or suspension of crude paliperidone free base in a solvent;
b) adjusting the pH of the first solution or suspension obtained in step-(a) to below 3.5 with hydrochloric acid to produce a second solution or suspension;
c) basifying the second solution or suspension obtained in step-(b) with a base by adjusting the pH to about 5 to 6 to produce a third solution or suspension;
d) combining the third solution or suspension with a reducing agent selected from the group consisting of sodium dithionite, sodium dithionate, sodium metabisulfite and potassium metabisulfite to produce a fourth solution;
e) optionally, subjecting the fourth solution to carbon treatment or silica gel treatment;
f) adjusting the pH of the fourth solution to about 8.5 to 9 with a base to produce a slurry containing paliperidone free base;
g) recovering paliperidone free base as a solid from the slurry obtained in step-(f);
h) combining the paliperidone solid obtained in step-(g) with water to form an aqueous slurry; and
i) isolating and/or recovering the highly pure paliperidone substantially free of the impurities from the aqueous slurry; and optionally converting the highly pure paliperidone obtained into a pharmaceutically acceptable salt thereof.

9. The process of claim 8, wherein the solvent used in step-(a) is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, and mixtures thereof; wherein the base used in steps-(c) and (f) is, each independently, an organic or inorganic base; and wherein the reducing agent in step-(d) is used directly or in the form of an aqueous solution or in the form of reducing agent dissolved in an organic solvent, wherein the organic solvent is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, and mixtures thereof.

10. The process of claim 9, wherein the solvent used in step-(a) is a mixture of water and methanol or isopropyl alcohol; wherein the organic base is selected from the group consisting of triethyl amine, trimethylamine, N,N-diisopropylethylamine, N-methylmorpholine and N-methylpiperidine; and wherein the inorganic base is selected from the group consisting of ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide.

11. The process of claim 8, wherein the pH of the first solution or suspension in step-(b) is adjusted between about 3 and 3.5; wherein the third solution or suspension obtained in step-(c) is optionally stirred at a temperature of about 25° C. to the reflux temperature of the solvent used; wherein the combining in step-(d) is accomplished by adding the third solution or suspension to the reducing agent or by adding the reducing agent to the third solution or suspension at a temperature of about 30° C. to about 90° C.; wherein the slurry obtained in step-(f) is optionally stirred at a temperature of about 15° C. to the reflux temperature of the solvent used for at least 15 minutes; wherein the recovering in step-(g) is accomplished by filtration, filtration under vacuum, decantation, centrifugation, filtration employing a filtration media of a silica gel or celite, or a combination thereof; wherein the isolation of highly pure paliperidone base in step-(i) is carried out by cooling the aqueous slurry while stirring at a temperature of below 30° C. for at least 15 minutes; wherein the solid obtained in step-(i) is recovered by filtration, filtration under vacuum, decantation, centrifugation, filtration employing a filtration media of a silica gel or celite, or a combination thereof; and wherein the pure paliperidone base obtained in step-(i) is optionally suction dried followed by suspending in an organic solvent, stirring the suspension at a temperature of about 30° C. to the reflux temperature of the organic solvent used and recovering the pure paliperidone, wherein the organic solvent is selected from the group consisting of an alcohol, a ketone, and mixtures thereof.

12. The process of claim 11, wherein the pH of the second solution or suspension in step-(c) is adjusted between about 5.5 and 6; wherein the reaction mass obtained after completion of the addition process in step-(d) is stirred at a temperature of about 50° C. to about 80° C. for about 10 minutes to about 10 hours; wherein the isolation in step-(i) is carried out by cooling the aqueous slurry while stirring at a temperature of about 0° C. to about 30° C. for about 30 minutes to about 20 hours; and wherein the pure paliperidone or a pharmaceutically acceptable salt thereof obtained in step-(i) is further dried under vacuum or at atmospheric pressure, at a temperature of about 35° C. to about 70° C.

13. A process for the preparation of pure paliperidone, comprising:
a) reacting 3-(2-chloro ethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole hydrochloride in the presence of a base, optionally in the presence of a metal iodide and a phase transfer catalyst, in a first solvent under atmospheric pressure or under high pressure of about 2 to 15 kg/cm² to produce a reaction mass containing crude paliperidone free base;
b) isolating paliperidone free base as a solid from the reaction mass obtained step-(a);
c) combining the solid paliperidone free base with water to form an aqueous slurry;
d) isolating paliperidone free base as a solid from the aqueous slurry;
e) dissolving the paliperidone free base obtained in step-(d) in a second solvent to produce a first solution;
f) combining the first solution with L-(+)-tartaric acid to produce a second solution or suspension;
g) isolating the solid state form of paliperidone L-(+)-tartrate salt from the second solution or suspension;
h) dissolving or suspending the paliperidone L-(+)-tartrate salt in a third solvent to produce a third solution or suspension;
i) combining the third solution or suspension with a reducing agent selected from the group consisting of sodium dithionite, sodium dithionate, sodium metabisulfite and potassium metabisulfite to produce a fourth solution or suspension;
j) adjusting the pH of the solution or suspension obtained in step-(h) or step-(i) to about 8 to 10 with a base to produce a fifth solution or suspension containing paliperidone free base; and
k) isolating and/or recovering the pure paliperidone free base from the fifth solution or suspension obtained in step-(j).

14. The process of claim 13, wherein the base used in any of the steps-(a) and (j), each independently, is an organic or inorganic base, wherein the organic base is selected from the group consisting of triethyl amine, trimethylamine, N,N-diisopropylethylamine, N-methylmorpholine and N-methylpiperidine, and wherein the inorganic base is selected from the group consisting of ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide; wherein the metal iodide employed in step-(a) is potassium iodide or sodium iodide; wherein the phase transfer catalyst is selected from the group consisting of tetrabutylammonium bromide, tetrabutylphosphonium bromide, tetrabutylammonium chloride, tetrabutylphosphonium chloride, benzyltriethylammonium chloride and tetrabutylammonium hydrogen sulfate; wherein the L-(+)-tartaric acid in step-(f) is used directly or in the form of an aqueous solution of L-(+)-tartaric acid or in the form of L-(+)-tartaric acid dissolved in an organic solvent, wherein the organic solvent is selected from the group consisting of methanol, ethanol, isopropyl alcohol, propanol, t-butanol, n-butanol, acetone, methyl ethyl ketone, methyl isobutyl ketone, diethyl ketone, tetrahydrofuran, N,N-dimethylformamide, N,N-dimethylacetamide, dimethylsulfoxide, and mixtures thereof; and wherein the reducing agent in step-(i) is used directly or in the form of an aqueous solution or in the form of reducing agent dissolved in an organic solvent, wherein the organic solvent is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, and mixtures thereof.

15. The process of claim 14, wherein the base used in the steps-(a) and (j), each independently, is selected from the group consisting of N,N-diisopropylethylamine, sodium carbonate and ammonia; and wherein the ammonia is used in the form of aqueous ammonia or in the form of ammonia dissolved in an organic solvent selected from the group consisting of an alcohol and a ketone.

16. The process of claim 13, wherein the first solvent used in step-(a) is selected from the group consisting of methanol, ethanol, isopropyl alcohol, propanol, t-butanol, n-butanol, acetone, methyl ethyl ketone, methyl isobutyl ketone, diethyl ketone, tetrahydrofuran, N,N-dimethylformamide, N,N-dimethylacetamide, dimethylsulfoxide, and mixtures thereof; wherein the second solvent used in step-(e) is selected from the group consisting of methanol, ethanol, isopropyl alcohol, propanol, t-butanol, n-butanol, acetone, methyl ethyl ketone, methyl isobutyl ketone, diethyl ketone, tetrahydrofuran, N,N-dimethylformamide, N,N-dimethylacetamide, dimethylsulfoxide, and mixtures thereof; and wherein the third solvent used in step-(h) is selected from the group consisting of water, methanol, ethanol, isopropyl alcohol, propanol, t-butanol, n-butanol, acetone, methyl ethyl ketone, methyl isobutyl ketone, diethyl ketone, methylene chloride, 1,2-dichloroethane, chloroform, carbon tetrachloride, n-pentane, n-hexane, n-heptane, cyclohexane, toluene, xylene acetonitrile, tetrahydrofuran, dimethylformamide, dimethylsulfoxide, and mixtures thereof.

17. The process of claim 16, wherein the first solvent is methanol; wherein the second solvent is N,N-dimethylformamide; and wherein the third solvent is a mixture of water and methanol.

18.-19. (canceled)

20. A process for the preparation of paliperidone intermediate, 3-(2-chloroethyl)-6,7,8,9-tetrahydro-9-hydroxy-2-methyl-4H-pyrido[1,2-a]-pyrimidin-4-one, comprising:
a) providing a solution of 3-(2-chloroethyl)-9-hydroxy-2-methyl-4H-pyrido[1,2-a]pyrimidin-4-one or an acid addition salt thereof in a first solvent;
b) optionally, subjecting the solution to carbon treatment or silica gel treatment;
c) hydrogenating the solution obtained in step-(a) or step-(b) in the presence of hydrogen gas using a hydrogenation catalyst to produce a reaction mass containing 3-(2-chloroethyl)-6,7,8,9-tetrahydro-9-hydroxy-2-methyl-4H-pyrido[1,2-a]-pyrimidin-4-one, where-in the hydrogenation catalyst is Pd/C/854-RD or Pd/C/213-RD;
d) isolating 3-(2-chloro ethyl)-6,7,8,9-tetrahydro-9-hydroxy-2-methyl-4H-pyrido[1,2-a]-pyrimidin-4-one as a solid from the reaction mass obtained in step-(c) using a second solvent;
e) combining the solid obtained in step-(d) with ethylenediaminetetraacetic acid (EDTA) or a salt thereof to produce a reaction mass; and
isolating and/or recovering substantially pure 3-(2-chloroethyl)-6,7,8,9-tetrahydro-9-hydroxy-2-methyl-4H-pyrido[1,2-a]-pyrimidin-4-one from the reaction mass obtained in step-(e).

21. The process of claim 20, wherein the first solvent used in step-(a) is selected from the group consisting of water, methanol, ethanol, n-propanol, isopropyl alcohol, isobutanol, n-butanol, tert-butanol, amyl alcohol, isoamyl alcohol, hexanol, and mixtures thereof; wherein the second solvent used in step-(d) is selected from the group consisting of water, methanol, ethanol, isopropyl alcohol, propanol, t-butanol, n-butanol, acetone, methyl ethyl ketone, methyl isobutyl ketone, diethyl ketone, and mixtures thereof; wherein the hydrogenation catalyst used in step-(c) is 10% Pd/C/213-RD or 10% Pd/C/854-RD; wherein the salt of ethylenediaminetetraacetic acid used in step-(e) is selected from the group consisting of mono sodium, disodium, trisodium and tetrasodium salts of ethylenediaminetetraacetic acid; and wherein the ethylenediaminetetraacetic acid or a salt thereof in step-(e) is used directly or in the form of an aqueous solution or in the form of ethylenediaminetetraacetic acid or a salt thereof dissolved in an organic solvent selected from the group consisting of an alcohol, a ketone, and mixtures thereof.

22.-24. (canceled)

25. A process for the preparation of 3-(2-chloroethyl)-9-hydroxy-2-methyl-4H-pyrido[1,2-a]pyrimidin-4-one hydrochloride salt, comprising:
a) reacting 2-amino-3-hydroxypyridine with 2-acetylbutyrolactone in the presence of a chlorinating agent to produce a reaction mass;
b) quenching the reaction mass in water to form a quenched reaction mass;
c) adjusting the pH of the quenched reaction mass to 4 to 6 with a base to produce a reaction mass containing 3-(2-chloro ethyl)-9-hydroxy-2-methyl-4H-pyrido[1,2-a]pyrimidin-4-one;
d) extracting the reaction mass obtained in step-(c) with methylene chloride to produce an organic layer;
e) adjusting the pH of the methylene chloride layer to below 2.0 by adding concentrated hydrochloric acid to produce an acidic reaction mass;
f) substantially removing the methylene chloride from the reaction mass obtained in step-(e) to form a residue;
g) combining the residue with an alcoholic solvent to produce a solution or suspension containing 3-(2-chloroethyl)-9-hydroxy-2-methyl-4H-pyrido[1,2-a]pyrimidin-4-one hydrochloride salt; and
h) isolating and/or recovering substantially pure 3-(2-chloroethyl)-9-hydroxy-2-methyl-4H-pyrido[1,2-a]pyrimidin-4-one hydrochloride salt from the solution or suspension obtained in step-(g).

26. The process of claim 25, wherein the chlorinating reagent used in step-(a) is selected from the group consisting of thionyl chloride, phosphorus oxychloride, phosphorus trichloride and phosphorus pentachloride; wherein the base used in step-(c) is an organic or inorganic base selected from the group consisting of triethyl amine, trimethylamine, N,N-diisopropylethylamine, N-methylmorpholine, N-methylpiperidine, ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide; and wherein the alcohol solvent used in step-(g) is selected from the group consisting of methanol, ethanol, isopropyl alcohol, propanol, t-butanol, n-butanol, and mixtures thereof.

27.-29. (canceled)

30. A process for the preparation of paliperidone 9-keto impurity, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione, comprising:
a) oxidizing paliperidone with oxalyl chloride in the presence of a base in a solvent to produce a reaction mass containing 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione; and
b) isolating 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione from the reaction mass obtained in step-(a).

31. (canceled)

32. The process of claim 30, wherein the solvent used in step-(a) is selected from the group consisting of methylene chloride, dimethylsulfoxide, and mixtures thereof; wherein the base used in step-(a) is triethyl amine; and wherein the reaction in step-(a) is carried out at a temperature of about −70° C. to about 0° C. for about 30 minutes to about 6 hours.

33. An isolated methanoyl compound, 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one, having of formula A:

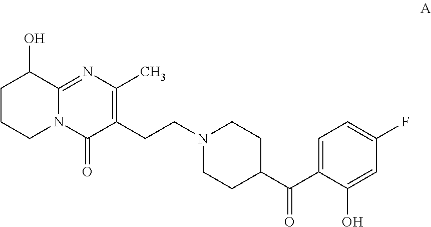

34. A process for the preparation of methanoyl compound of claim 33, comprising:
a) hydrogenating paliperidone using hydrogen gas in the presence of a hydrogenation catalyst and a base in a first solvent to produce a reaction mass containing 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one; and
b) isolating 3-[2-[4-[1-(4-fluoro-2-hydroxyphenyl)methanoyl]piperidinyl-1-yl]ethyl]-2-methyl-6,7,8,9-tetrahydro-4H-pyrido[1,2-a]pyrimidin-4-one from the reaction mass obtained in step-(a) using a second solvent.

35. (canceled)

36. The process of claim 34, wherein the first solvent used in step-(a) is a mixture of water and methanol; wherein the second solvent used in step-(b) is a mixture of methylene chloride and methanol; wherein the hydrogenation reaction is carried out at a temperature of about 20° C. to about 35° C. for about 2 hours to about 5 hours; and wherein the base used in step-(a) is sodium hydroxide.

37. An isolated dehydroxy compound, 3-[2-[4-(6-fluoro-1,2-benzisoxazol-3-yl)-1-piperidinyl]ethyl]-2-methyl-4H-pyrido[1,2-a]pyrimidin-4-one, of formula B:

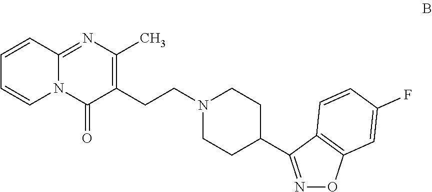

38. A process for the preparation of paliperidone dehydroxy compound of claim 37, comprising:
a) reacting 3-(2-chloroethyl)-2-methyl-4H-pyrido[1,2-a]pyrimidin-4-one with 6-fluoro-3-(4-piperidinyl)-1,2-benzisoxazole hydrochloride in the presence of N,N-diisopropylethylamine, sodium iodide and tetrabutylammonium bromide in a first solvent to produce a reaction mass containing 3-[2-[4-(6-fluoro-1,2-benzisoxazol-3-yl)-1-piperidinyl]ethyl]-2-methyl-4H-pyrido[1,2-a]pyrimidin-4-one; and
b) isolating 3-[2-[4-(6-fluoro-1,2-benzisoxazol-3-yl)-1-piperidinyl]ethyl]-2-methyl-4H-pyrido[1,2-a]pyrimidin-4-one from the reaction mass obtained in step-(a) using a second solvent.

39. (canceled)

40. The process of claim 38, wherein the first solvent used in step-(a) is methanol; wherein the second solvent used in step-(b) is a mixture of water and methanol; and wherein the condensation reaction in step-(a) is carried out at about 50° C. to about 70° C. for about 20 hours to about 75 hours.

41. The highly pure paliperidone or a pharmaceutically acceptable salt thereof of claim 1, further comprising one or more pharmaceutically acceptable excipients to form a pharmaceutical composition.

42. The pharmaceutical composition of claim 41, wherein the paliperidone or a pharmaceutically acceptable salt thereof has a D90 particle size of less than or equal to about 400 microns.

43. The pharmaceutical composition of claim 42, wherein the D₉₀ particle size is about 1 micron to about 300 microns, or about 10 microns to about 150 microns.

44. (canceled)
